SEQUENCE LISTING (15 sequences)

SEQ ID NO: 1 - DNA, 29 bp, Homo sapiens
agagacgagattgtctctcaaaatgatgt

SEQ ID NO: 2 - DNA, 31 bp, Homo sapiens
tctaatctaagcctaagttttcctaacagaa

SEQ ID NO: 3 - DNA, 30 bp, Homo sapiens
ggactacagcaatggtgagggagctgtttt

SEQ ID NO: 4 - DNA, 30 bp, Homo sapiens
ctatgcattcattgcattcacagcttcatg

SEQ ID NO: 5 - DNA, 30 bp, Homo sapiens
aggcccaagaatatgggcacacatccttca

SEQ ID NO: 6 - DNA, 31 bp, Homo sapiens
actgctcgacagcatggatgttccctgcaca

SEQ ID NO: 7 - DNA, 30 bp, Homo sapiens
catgaaaggtatagaaccctggtatgcttt

SEQ ID NO: 8 - DNA, 30 bp, Homo sapiens
aactaatgtaagatgagctaaccttgctct

SEQ ID NO: 9 - DNA, 30 bp, Homo sapiens
actgatgtggagcgacagatgaccgcacag

SEQ ID NO: 10 - DNA, 30 bp, Homo sapiens
attgtcaaataacacgaaagtggtgaaaac

SEQ ID NO: 11 - DNA, 30 bp, Homo sapiens
gggatgaaaagcactcttgagaaaggcatg

SEQ ID NO: 12 - DNA, 30 bp, Homo sapiens
ataaagaaaatgtgatacatatacaccatg

SEQ ID NO: 13 - protein, 1244 aa, Homo sapiens
MNNQKVVAVLLQECKQVLDQLLLEAPDVSEEDKSEDQRCRALLPSELRTLIQEAKEMKWPFVPEKWQYKQAVGPEDKTNLKDVIGAGLQQLLASLRASILARDCAAAAAIVFLVDRFLYGLDVSGKLLQVAKGLHKLQPATPIAPQVVIRQARISVNSGKLLKAEYILSSLISNNGATGTWLYRNESDKVLVQSVCIQIRGQILQKLGMWYEAAELIWASIVGYLALPQPDKKGLSTSLGILADIFVSMSKNDYEKFKNNPQINLSLLKEFDHHLLSAAEACKLAAAFSAYTPLFVLTAVNIRGTCLLSYSSSNDCPPELKNLHLCEAKEAFEIGLLTKRDDEPVTGKQELHSFVKAAFGLTTVHRRLHGETGTVHAASQLCKEAMGKLYNFSTSSRSQDREALSQEVMSVIAQVKEHLQVQSFSNVDDRSYVPESFECRLDKLILHGQGDFQKILDTYSQHHTSVCEVFESDCGNNKNEQKDAKTGVCITALKTEIKNIDTVSTTQEKPHCQRDTGISSSLMGKNVQRELRRGGRRNWTHSDAFRVSLDQDVETETEPSDYSNGEGAVFNKSLSGSQTSSAWSNLSGFSSSASWEEVNYHVDDRSARKEPGKEHLVDTQCSTALSEELENDREGRAMHSLHSQLHDLSLQEPNNDNLEPSQNQPQQQMPLTPFSPHNTPGIFLAPGAGLLEGAPEGIQEVRNMGPRNTSAHSRPSYRSASWSSDSGRPKNMGTHPSVQKEEAFEIIVEFPETNCDVKDRQGKEQGEEISERGAGPTFKASPSWVDPEGETAESTEDAPLDFHRVLHNSLGNISMLPCSSFTPNWPVQNPDSRKSGGPVAEQGIDPDASTVDEEGQLLDSMDVPCTNGHGSHRLCILRQPPGQRAETPNSSVSGNILFPVLSEDCTTTEEGNQPGNMLNCSQNSSSSSVWWLKSPAFSSGSSEGDSPWSYLNSSGSSWVSLPGKMRKEILEARTLQPDDFEKLLAGVRHDWLFQRLENTGVFKPSQLHRAHSALLLKYSKKSELWTAQETIVYLGDYLTVKKKGRQRNAFWVHHLHQEEILGRYVGKDYKEQKGLWHHFTDVERQMTAQHYVTEFNKRLYEQNIPTQIFYIPSTILLILEDKTIKGCISVEPYILGEFVKLSNNTKVVKTEYKATEYGLAYGHFSYEFSNHRDVVVDLQGWVTGNGKGLIYLTDPQIHSVDQKVFTTNFGKRGIFYFFNNQHVECNEICHRLSLTRPSMEKPCT

SEQ ID NO: 14 - protein, 550 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRKGKQTRQQQEHQKYMVPLQASAQEKNGL

SEQ ID NO: 15 - protein, 563 aa, Homo sapiens
MTFSEILDRVGSMGHFQFLHVAILGLPILNMANHNLLQIFTAATPVHHCRPPHNASTGPWVLPMGPNGKPERCLRFVHPPNASLPNDTQRAMEPCLDGWVYNSTKDSIVTEWDLVCNSNKLKEMAQSIFMAGILIGGLVLGDLSDRFGRRPILTCSYLLLAASGSGAAFSPTFPIYMVFRFLCGFGISGITLSTVILNVEWVPTRMRAIMSTALGYCYTFGQFILPGLAYAIPQWRWLQLTVSIPFFVFFLSSWWTPESIRWLVWKVLEGPEDTPAGGCLQWQEEGERLSLEELKLNLQKEISLAKAKYTASDLFRIPMLRRMTFCLSLAWFATGFAYYSLAMGVEEFGVNLYILQIIFGGVDVPAKFITILSLSYLGRHTTQRCPAPGRAILALTFVPLDLQTVRTVLAVFGKGCLSSSFSCLFLYTSELYPTVIRQTGMGVSNLWTRVGSMVSPLVKITGEVQPFIPNIIYGITALLGGSAALFLPETLNPCQRLSKTWKTGQSLPLAPSVLLPGEAGLGPGLFLSSLSLGRAKKPKQEPEVEKASQRIPLQPHGPGLGSS

(19) United States
(12) Patent Application Publication
(10) Pub. No.: US 2009/0098056 A1
(43) Pub. Date: Apr. 16, 2009

(54) ALPK1 GENE VARIANTS IN DIAGNOSIS RISK OF GOUT

(75) Inventors: Ying-Chin Ko, Kaohsiung City (TW); Shih-Feng Tsai, Miaoli County (TW)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: National Health Research Institute, Zhunan (TW)

(21) Appl. No.: 12/207,914

(22) Filed: Sep. 10, 2008

Related U.S. Application Data
(60) Provisional application No. 60/993,136, filed on Sep. 10, 2007.

(51) Int. Cl. A61B 10/00; C12Q 1/68; G01N 33/566; C07H 21/04; C40B 40/06; C40B 30/04; C12N 15/85; C12N 5/10

(52) U.S. Cl. 424/9.2; 435/6; 435/7.8; 536/23.1; 506/16; 506/9; 435/320.1; 435/325

(57) ABSTRACT

The present invention relates to a method of identifying a human subject having an elevated risk of gout and/or hyperuricemia by detecting the occurrence of at least one SNP associated with an elevated risk of gout in an ALPK1 gene in a biological sample from the subject, or by determining the expression level of an ALPK1 gene in a biological sample from the subject. Also disclosed is an isolated nucleic acid molecule, its complement or gene variant comprising at least one of the polymorphisms identified herein to be associated with gout and/or hyperuricemia, a kit for performing a diagnostic test to identify a human subject having an elevated risk of gout and/or hyperuricemia, and a method of selecting or identifying a compound useful for treating gout and/or hyperuricemia.

### CROSS REFERENCE TO RELATED APPLICATIONS

This application claims the benefit of U.S. Provisional Application No. 60/993,136 filed Sep. 10, 2007, which is herein incorporated by reference in its entirety.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 362547SequenceListing.txt, a creation date of Sep. 10, 2008, and a size of 22 KB. The sequence listing filed via EFS-Web is part of the specification and is hereby incorporated in its entirety by reference herein.

### BACKGROUND OF THE INVENTION

Gout is a rheumatic disease characterized by inflammatory arthritis or elevated serum urate level. As the amount of serum urate or uric acid exceeds its physiologic solubility limit, it crystallizes as monosodium urate on the articular cartilage of joints, tendons and surrounding tissues. The intra-articular crystal deposition and tissue accumulation of monosodium urate monohydrate may trigger recurrent attacks and provoke an inflammatory reaction of these tissues.

Hyperuricemia is a common feature of gout. Hyperuricemia is defined as serum uric acid level of more than 7.0 mg/dL in men or more than 6.0 mg/dL in women and can be caused by either overproduction or underexcretion of uric acid. In the general population, the vast majority of gout patients (>90%) suffer from underexcretion. Hyperuricemia has known associations with hypertension, hyperlipidemia, diabetes, obesity and coronary artery disease.

A high prevalence of gout and hyperuricemia in Taiwan aborigines and other Pacific Austronesian populations suggests a possible founder effect across the Pacific. Gout may be genetically linked as found in some rare forms of Mendelian disorders, e.g. hypoxanthine guanine phosphoribosyltransferase (HPRT) for X-linked gout, Autosomal Dominant Medullary Cystic Disease on chromosome 1q21, or Familial Juvenile Hyperuricemia Nephropathy on chromosome 16 (Reginato et al., Curr Opin Rheumatol 19: 134-145 (2007)). A previous study conducted by Ko et al., described in U.S. Patent Application Number US 2005/0170387A1, has determined a gout related gene locus on chromosome 4q25.

There are a number of other factors known to raise an individual's risk of having gout and/or hyperuricemia. Some of these include: high purine diet, alcohol intake, age and gender. Epidermiologic studies have shown that both environmental factors and genetic predisposition contribute to high serum uric acid levels in the occurrence of hyperuricemia or gout (Choi et al., N Eng J Med 350: 1093-1103 (2004); Choi et al., Lancet 363: 1277-1281 (2004)). In terms of genetic regulation of uric acid transport in kidney, several molecular candidates have also been proposed for the pathway of urate absorption, secretion and excretion, including OATs, URAT1, galectin-9, OATV1, MRP4 (Hediger et al., Physiology (Bethesda) 20: 125-133 (2005); Choi et al., Ann Intern Med 143: 499-516 (2005)).

There is an ongoing need in the art for identifying genetic markers that are linked to hyperuricemia and an elevated risk of gout. The present invention provides such new genetic markers and their uses in the development of diagnosis and therapeutic strategies for targeted populations having an elevated risk of gout and/or hyperuricemia.

### BRIEF SUMMARY OF THE INVENTION

In one aspect, the present invention relates to a method of identifying a human subject having an elevated risk of gout and/or hyperuricemia. The method comprises detecting the occurrence of at least one single nucleotide polymorphism (SNP) in an ALPK1 gene in a biological sample from the human subject. The at least one SNP is selected from the group consisting of a polymorphism comprising “C” at the nucleotide corresponding to nucleotide 15 of SEQ ID NO:1 (rs916868) or “G” at the nucleotide corresponding to nucleotide 15 of the corresponding minus strand of SEQ ID NO:1; a polymorphism comprising “G” at the nucleotide corresponding to nucleotide 15 of SEQ ID NO:2 (rs9994944) or “C” at the nucleotide corresponding to nucleotide 17 of the corresponding minus strand of SEQ ID NO:2; a polymorphism comprising “A” at the nucleotide corresponding to nucleotide 15 of SEQ ID NO:3 (rs2074388) or “T” at the nucleotide corresponding to nucleotide 16 of the corresponding minus strand of SEQ ID NO:3; a polymorphism comprising “G” at the nucleotide corresponding to nucleotide 16 of SEQ ID NO:4 (rs13148353) or “C” at the nucleotide corresponding to nucleotide 15 of the corresponding minus strand of SEQ ID NO:4; a polymorphism comprising “A” at the nucleotide corresponding to nucleotide 15 of SEQ ID NO:5 (rs2074379) or “T” at the nucleotide corresponding to nucleotide 16 of the corresponding minus strand of SEQ ID NO:5; a polymorphism comprising “C” at the nucleotide corresponding to nucleotide 15 of SEQ ID NO:6 (rs11726117) or “G” at the nucleotide corresponding to nucleotide 17 of the corresponding minus strand of SEQ ID NO:6; a polymorphism comprising “C” at the nucleotide corresponding to nucleotide 16 of SEQ ID NO:7 (rs6841595) or “G” at the nucleotide corresponding to nucleotide 15 of the corresponding minus strand of SEQ ID NO:7; a polymorphism comprising “T” at the nucleotide corresponding to nucleotide 15 of SEQ ID NO:8 (rs11098156) or “A” at the nucleotide corresponding to nucleotide 16 of the corresponding minus strand of SEQ ID NO:8; a polymorphism comprising “G” at the nucleotide corresponding to nucleotide 15 of SEQ ID NO:9 (rs231247) or “C” at the nucleotide corresponding to nucleotide 16 of the corresponding minus strand of SEQ ID NO:9; a polymorphism comprising “A” at the nucleotide corresponding to nucleotide 16 of SEQ ID NO:10 (rs55840220) or “T” at the nucleotide corresponding to nucleotide 15 of the corresponding minus strand of SEQ ID NO:10; a polymorphism comprising “G” at the nucleotide corresponding to nucleotide 16 of SEQ ID NO:11 (rs231253) or “C” at the nucleotide corresponding to nucleotide 15 of the corresponding minus strand of SEQ ID NO:11; and a polymorphism comprising “A” at the nucleotide corresponding to nucleotide 15 of SEQ ID NO:12 (rs960583) or “T” at the nucleotide corresponding to nucleotide 16 of the corresponding minus strand of SEQ ID NO:12; wherein the occurrence of the at least one SNP is indicative of an elevated risk of gout and/or hyperuricemnia in the human subject.

In certain embodiments of this aspect of the invention, the identification method comprises detecting the occurrence of at least two SNPs selected from the group of SNPs described above in an ALPK1 gene in a biological sample from the human subject.

In accordance with another aspect of the invention, the present invention relates to a method of identifying a human subject having an elevated risk of gout and/or hyperuricemia by determining the expression level of an ALPK1 gene in a biological sample from the human subject, wherein an elevated expression level of an ALPK1 gene is indicative of an elevated risk of gout in the human subject. According to certain examples of the invention, the expression level of ALPK1 gene is determined by real-time reverse transcription polymerase chain reaction (qRT-PCR).

In another aspect, the present invention relates to a kit for performing a diagnostic test to identify a human subject having an elevated risk of gout and/or hyperuricemia. The kit comprises at least one pair of primers designed for PCR amplification of a target polynucleotide sequence that comprises a SNP in an ALPK1 gene in a biological sample from a human subject, wherein the SNP is selected from the group of SNPs described above, and instructions for performing the diagnostic test.

In accordance with another aspect of the invention, the kit comprises at least one pair of primers designed for determination of the expression level of an ALPK1 gene in a biological sample from a human subject by qRT-PCR, and instructions for performing the diagnostic test.

In another aspect, the present invention relates to a method of selecting a compound useful for treating gout and/or hyperuricemia. The method comprises contacting a test compound in a buffering solution with a first polypeptide comprising a catalytic domain of an ALPK1 protein and a second polypeptide comprising a phosphorylation site for the ALPK1 protein on an OAT 1 protein, OAT 3 protein or OAT4L (URAT1) protein; detecting a change in phosphorylation level of the second polypeptide as a result of the phosphorylation of the second polypeptide by the first polypeptide; and selecting the test compound by its ability to decrease the phosphorylation level as compared to a control measurement wherein only the buffering solution, and not the test compound, is contacted with the first and second polypeptides. According to one example of the invention, the first polypeptide comprises an amino acid sequence of SEQ ID NO:13. As another example of the invention, the second polypeptide comprises an amino acid sequence of SEQ ID NO:14 or SEQ ID NO:15. According to other examples of the invention, the method further comprises administering the test compound to an animal model and determining the effect of the test compound on a symptom related to gout and/or hyperuricemia in the animal model.

In another aspect, the present invention relates to a method of identifying a compound useful for treating gout and/or hyperuricemia. The method comprises contacting a test compound with a host cell that expresses a gene operably linked to a regulatory sequence for an ALPK1 gene; detecting a change in the expression level of the gene from the host cell; and selecting the test compound by its ability to decrease the expression level of the gene, as compared to a control measurement wherein only the buffering solution, not the test compound, is contacted with the host cell. As one example of the invention, the gene may be a reporter gene operably linked to the regulatory sequence for ALPK1 gene. The reporter gene may encode a protein selected from the group consisting of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase. In other examples of the invention, the method further comprises administering the test compound to an animal model and determining the effect of the test compound on a symptom related to gout and/or hyperuricemia, in the animal model.

In another aspect, the present invention relates to an isolated nucleic acid molecule comprising a SNP of an ALPK1 gene, wherein the SNP is selected from the group of SNPs described above.

In addition, the present invention further relates to a vector comprising the isolated nucleic acid molecule. The present invention also relates to a host cell comprising the vector.

### BRIEF DESCRIPTION OF THE DRAWINGS AND TABLES

The foregoing summary, as well as the following detailed description of the invention, will be better understood when read in conjunction with the appended drawings and tables. For the purpose of illustrating the invention, there are shown in the drawings embodiments which are presently preferred. It should be understood, however, that the invention is not limited to the precise arrangements and instrumentalities shown.

In the drawings:

FIG. 1 illustrates haplotype analysis and a linkage disequilibrium (LD) plot for 11 major SNPs in a 16.7 kb region of an ALPK1 gene; and

FIG. 2 is a histogram showing ratios of ALPK1 mRNA levels between non-gout (Controls) and gout groups, between non-gout having AA homozygote (Controls/AA) and gout having GG homozygote, and between genotypes AA, AG and GG at a SNP position (NCBI dbSNP ID: rs231247) in the mixed subjects.

In the tables:

Table 1 shows results of multiple linkage analyses by fine mapping for gout using markers and/or SNPs in the 4q25 region;

Table 2 shows results of association of ALPK1 gene SNPs with gout susceptibility in family-based and population-based gout cases and controls;

Table 3 shows results of association of ALPK1 gene SNPs in hyperuricemia cases and controls;

Table 4 shows results of family-based and population-based combined association analysis for ALPK1 in gout cases and controls;

Table 5 shows independent and combined effects of ALPK1 variant and alcohol consumption by genotype with gout risk;

Table 6 shows independent and combined effects of ALPK1 variant and alcohol consumption by allele with gout risk;

Table 7 shows association results of haplotype analysis across ALPK1 constructed in gout cases and controls; and

Table 8 shows the association of the SNP (rs231247) with uric acid levels and fractional excretion of uric acid (FEUA).

### DETAILED DESCRIPTION OF THE INVENTION

### Definitions

As used herein, the term “allele” refers to one of several alternative forms of a nucleotide sequence that may occupy a locus or position on a chromosome. An “allele” can be a DNA sequence for a gene. An “allele” can also be a non-gene sequence. A diploid organism or cell has two copies of each chromosome. If the same allele occupies corresponding loci on the pair of chromosomes, the organism or cell is homozygous for this allele. If the different alleles occupy corresponding loci on the pair of chromosomes, the organism or cell is heterozygous for both alleles.

As used herein, the term “gene” refers to a segment of DNA involved in producing a peptide, polypeptide, or protein, and the mRNA encoding such protein species, including the coding region, non-coding regions preceding (“5′UTR”) and following (“3′UTR”) the coding region. A “gene” may also include intervening non-coding sequences (“introns”) between individual coding segments (“exons”). A “coding region” or “coding sequence” refers to the portion of a gene that encodes amino acids and the start and stop signals for the translation of the corresponding polypeptide via triplet-base codons.

As used herein, a “regulatory sequence” refers to the portion of a gene that can control the expression of the gene. A “regulatory sequence” can include promoters, enhancers and other expression control elements such as polyadenylation signals, ribosome binding site (for bacterial expression), and/or an operator. As used herein, the term “promoter” means a regulatory sequence of DNA that is involved in the binding of RNA polymerase to initiate transcription of a gene. Promoters are often upstream (“5′ to”) the transcription initiation site of the gene. An “enhancer” means a regulatory sequence of DNA that can regulate the expression of a gene in a distance- and orientation-dependent fashion.

As used herein, an “ALPK1 gene” refers to a gene involved in producing an ALPK1 protein. An ALPK1 gene can be a genomic sequence or a complementary DNA (cDNA) sequence synthesized from a mature mRNA by reverse transcription.

As used herein an “ALPK1 protein”, also known as “alpha-kinase 1”, “lymphocyte alpha-kinase” refers to a protein that has a protein kinase activity, recognizing and phosphorylating another protein at a phosphorylation site in which the surrounding peptides have an alpha-helical conformation; and that i) has greater than about 80% amino acid sequence identity to a human ALPK1 protein having an amino acid sequence of SEQ ID NO:13 (GenBank protein Id. NP_{—}079420), or ii) binds to antibodies, e.g., polyclonal or monoclonal antibodies, raised against a human ALPK1 protein having an amino acid sequence of SEQ ID NO:13.

A “reporter gene” refers to a nucleic acid sequence that encodes a reporter gene product. As is known in the art, reporter gene products are typically easily detectable by standard methods. Exemplary suitable reporter genes include, but are not limited to, genes encoding luciferase (lux), β-galactosidase (lacZ), green fluorescent protein (GFP), chloramphenicol acetyltransferase (CAT), β-glucuronidase, neomycin phosphotransferase, and guanine xanthine phosphoribosyl-transferase proteins.

The term “biological sample” refers to a sample obtained from an organism (e.g., patient) or from components (e.g., cells) of an organism. The sample may be of any biological tissue, cell(s) or fluid. The sample may be a “clinical sample” which is a sample derived from a patient. Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.” A “biological sample” may also include a substantially purified or isolated protein, membrane preparation, or cell culture.

As used herein, the term “polymorphism” or “polymorphic” refers to the occurrence of allelic variations at a particular locus or position among individuals in a population. The polymorphic region or polymorphic site refers to an amplified region of the nucleic acid where the nucleotide difference that distinguishes the alleles occurs.

As used herein, a “single nucleotide polymorphism” or “SNP” refers to a polymorphism wherein the polymorphic site consists of a single nucleotide position. SNP involves the naturally occurring substitution of a single nucleotide at a given location in the genomes of members of a species or between paired chromosomes in an individual. For example, two corresponding amplified DNA fragments are sequenced from different individuals. One amplified DNA fragment contains C at a particular position, and the other contains T at the corresponding position. These two amplified DNA fragments carry two different alleles of a SNP at that particular position. Often, there are only two alleles at a SNP position, such as C and T.

As used herein, the term “allele frequency” refers to a measure of the relative frequency of an allele on a genetic locus in a population. As used herein, a “major SNP” refers to a SNP having an allele frequency of 50% or more; and a “minor SNP” refers to a SNP having an allele frequency of less than 50%.

SNPs may fall within coding sequences of genes, noncoding sequences of genes, or in the intergenic regions between genes. SNPs within a coding sequence will not necessarily change the amino acid sequence of the polypeptide that is encoded, due to degeneracy of the genetic code. SNPs that change the amino acid sequence of the encoded polypeptide will not necessarily change the phenotype of the organism or the cell. SNPs that are not in coding regions may still have consequences for gene splicing, transcription factor binding, or the sequence of non-coding RNA. Comparing SNPs between organisms of a species with and without a disease, may lead to useful biomedical information about the organisms, such as how they develop diseases, respond to pathogens, chemicals, drugs, etc.

As used herein, “elevated risk of gout” refers to a greater likelihood of having or developing gout in an individual having a genetic predisposition, compared to an individual that does not have that genetic predisposition. Examples of the genetic predisposition, include, but are not limited to, having a particular allele, such as a particular nucleotide at a particular SNP, or having an altered expression level of a particular gene, such as increased expression of an ALPK1 gene. And “hyperuricemia” was defined as a serum uric acid level of more than 7.0 mg/dL in men or more than 6.0 mg/dL in women.

As used herein, “amplification” when used in the context of a polynucleotide sequence refers to any means by which the polynucleotide sequence is copied and thus expanded into a larger number of polynucleotide sequences, e.g., by reverse transcription, polymerase chain reaction, and ligase chain reaction.

As used herein, the term “instructions” when used in the context of a kit includes a writing, a recording, a diagram, an analog or digital electronic medium or any other medium of expression which can be used to communicate the usefulness of the kit for its designated use. The instructions may, for example, be affixed to or included within a container for the kit.

In the context of the present invention, adenosine is abbreviated as “A”, cytidine is abbreviated as “C”, guanine is abbreviated as “G”, thymidine is abbreviated as “T”, and uridine is abbreviated as “U”.

As used herein, the term “nucleotide sequence”, “nucleic acid” or “polynucleotide” refers to the arrangement of either deoxyribonucleotide or ribonucleotide residues in a polymer in either single- or double-stranded form. Nucleic acid sequences can be composed of natural nucleotides of the following bases: T, A, C, G, and U, and/or synthetic analogs of the natural nucleotides.

As used herein, an “isolated” nucleic acid molecule is one that is substantially separated from at least one of the other nucleic acid molecules present in the natural source of the nucleic acid, or is substantially free of at least one of the chemical precursors or other chemicals when the nucleic acid molecule is chemically synthesized. An “isolated” nucleic acid molecule can also be, for example, a nucleic acid molecule that is substantially free of at least one of the nucleotide sequences that naturally flank the nucleic acid molecule at its 5′ and 3′ ends in the genomic DNA of the organism from which the nucleic acid is derived. A nucleic acid molecule is “substantially separated from” or “substantially free of” other nucleic acid molecule(s) or other chemical(s) in preparations of the nucleic acid molecule when there is less than about 30%, 20%, 10%, or 5% or less, and preferably less than 1%, (by dry weight) of the other nucleic acid molecule(s) or the other chemical(s) (also referred to herein as a “contaminating nucleic acid molecule” or a “contaminating chemical”).

Isolated nucleic acid molecules include, without limitation, separate nucleic acid molecules (e.g., cDNA or genomic DNA fragments produced by PCR or restriction endonuclease treatment) independent of other sequences, as well as nucleic acid molecules that are incorporated into a vector, an autonomously replicating plasmid, a virus (e.g., a retrovirus, adenovirus, or herpes virus), or into the genomic DNA of a prokaryote or eukaryote. In addition, an isolated nucleic acid molecule can include a nucleic acid molecule that is part of a hybrid or fusion nucleic acid molecule. An isolated nucleic acid molecule can be a nucleic acid sequence that is: (i) amplified in vitro by, for example, polymerase chain reaction (PCR); (ii) synthesized by, for example, chemical synthesis; (iii) recombinantly produced by cloning; or (iv) purified, as by cleavage and electrophoretic or chromatographic separation.

A polynucleotide can have a single strand or parallel and anti-parallel strands. Thus, a polynucleotide may be a single-stranded or a double-stranded nucleic acid. A polynucleotide is not defined by length and thus includes very large nucleic acids, as well as short ones, such as an oligonucleotide.

Conventional notation is used herein to describe polynucleotide sequences. The left-hand end of a single-stranded polynucleotide sequence is the 5′-end, and the left-hand direction of a single-stranded polynucleotide sequence is referred to as the 5′-direction. The left-hand end of a double-stranded polynucleotide sequence is the 5′-end of the plus strand, which is depicted as the top strand of the double strands, and the right-hand end of the double-stranded polynucleotide sequence is the 5′-end of the minus strand, which is depicted as the bottom strand of the double strands. The direction of 5′ to 3′ addition of nucleotides to nascent RNA transcripts is referred to as the transcription direction. A DNA strand having the same sequence as an mRNA is referred to as the “coding strand.” Sequence on a DNA strand which is located 5′ to a reference point on the DNA is referred to as “upstream sequence”; sequence on a DNA strand which is 3′ to a reference point on the DNA is referred to as “downstream sequence.”

As used herein, “nucleotide X of a nucleotide sequence” refers to the nucleotide that is the Xth residue of the nucleotide sequence counting from its 5′ end. For example, “nucleotide 15 of SEQ ID NO: 1” refers to the 15^{th} residue of SEQ ID NO:1 counting from its 5′ end.

The term “oligonucleotide” refers to a DNA or RNA sequence of a relatively short length, for example, less than 100 residues long, generally no greater than about 50 nucleotides. For many methods, oligonucleotides of about 16-25 nucleotides in length are useful, although longer oligonucleotides of greater than about 25 nucleotides may sometimes be utilized. Some oligonucleotides can be used as “primers” for the synthesis of complimentary nucleic acid strands. Oligonucleotides are also useful as “probes” for hybridization in several methods of nucleic acid detection, for example, in Northern blotting or in situ hybridization.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. Typical uses of primers include, but are not limited to, sequencing reactions and amplification reactions. A primer is typically single-stranded, but may be double-stranded. Primers are typically deoxyribonucleic acids, but a wide variety of synthetic and naturally-occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., detectable moieties, such as chromogenic, radioactive or fluorescent moieties, or moieties for isolation, e.g., biotin.

A “probe” or “detection probe” may be designed for the detection of a target polynucleotide sequence, such as that comprises a SNP. In one embodiment, the detection probe hybridizes to the target polynucleotide sequence comprising a SNP only if a particular SNP allele is present and the detection probe hybridizes to the target polynucleotide sequence at a sequence that straddles the SNP position. In another embodiment, the detection probe hybridizes to the target polynucleotide sequence at a sequence that does not straddle the SNP position, but is 3′ to and zero, one, or several nucleotides removed from the SNP position, provided that between the SNP position and the position where the 3′ end of the probe annealed to, the target polynucleotide sequence does not contain a nucleotide of the same type as the nucleotide at the SNP position.

In preferred embodiments, a primer or a probe hybridizes to a respective template or target polynucleotide sequence under stringent hybridization conditions. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6× sodium chloride/sodium citrate (SSC) at about 45° C., followed by one or more washes in 0.2×SSC and 0.1% SDS at 50-65° C., depending upon the length over which the hybridizing polynucleotides share complementarity.

As used herein, the terms “polypeptide” and “protein” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide” and “protein” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by the codons of genes may also be included in a polypeptide.

An “isolated protein” or “isolated polypeptide” is one that is substantially separated from at least one of the other proteins present in the natural source of the protein, or is substantially free of at least one of the chemical precursors or other chemicals when the protein is chemically synthesized. A protein is “substantially separated from” or “substantially free of” other protein(s) or other chemical(s) in preparations of the protein when there is less than about 30%, 20%, 10%, or 5% or less, and preferably less than 1%, (by dry weight) of the other protein(s) or the other chemical(s) (also referred to herein as a “contaminating protein” or a “contaminating chemical”).

Isolated proteins can have several different physical forms. The isolated protein can exist as a full-length nascent or unprocessed polypeptide, or as a partially processed polypeptide or as a combination of processed polypeptides. The full-length nascent polypeptide can be postranslationally modified by specific proteolytic cleavage events that result in the formation of fragments of the full-length nascent polypeptide. A fragment, or physical association of fragments can have the biological activity associated with the full-length polypeptide; however, the degree of biological activity associated with individual fragments can vary.

An isolated polypeptide or isolated protein can be a non-naturally occurring polypeptide. For example, an isolated polypeptide can be a “hybrid polypeptide.” An isolated polypeptide can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemical synthesis, as will be apparent to skilled artisans.

As used herein, “recombinant” refers to a polynucleotide, a polypeptide encoded by a polynucleotide, a cell, a viral particle or an organism that has been modified using molecular biology techniques to something other than its natural state.

As used herein, a “recombinant cell” or “recombinant host cell” is a cell that has had introduced into it a recombinant polynucleotide sequence. For example, recombinant cells can contain at least one nucleotide sequence that is not found within the native (non-recombinant) form of the cell or can express native genes that are otherwise abnormally expressed, under-expressed, or not expressed at all. Recombinant cells can also contain genes found in the native form of the cell wherein the genes are modified and re-introduced into the cell by artificial means. The term also encompasses cells that contain an endogenous nucleic acid that has been modified without removing the nucleic acid from the cell; such modifications include those obtained, for example, by gene replacement, and site-specific mutation.

Recombinant DNA sequence can be introduced into host cells using any suitable method including, for example, electroporation, calcium phosphate precipitation, microinjection, transformation, biolistics and viral infection. Recombinant DNA may or may not be integrated (covalently linked) into chromosomal DNA making up the genome of the cell. For example, the recombinant DNA can be maintained on an episomal element, such as a plasmid. Alternatively, with respect to a stably transformed or transfected cell, the recombinant DNA has become integrated into the chromosome so that it is inherited by daughter cells through chromosome replication. This stability is demonstrated by the ability of the stably transformed or transfected cell to establish cell lines or clones comprised of a population of daughter cells containing the exogenous DNA. Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila- and silkworm-derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

As used herein, “operably linked”, refers to a functional relationship between two nucleotide sequences. A single-stranded or double-stranded nucleic acid moiety comprises the two nucleotide sequences arranged within the nucleic acid moiety in such a manner that at least one of the two nucleotide sequences is able to exert a physiological effect by which it is characterized upon the other. By way of example, a promoter sequence that controls expression (for example, transcription) of a coding sequence is operably linked to that coding sequence. Operably linked nucleic acid sequences can be contiguous, typical of many promoter sequences, or non-contiguous, in the case of, for example, nucleic acid sequences that encode repressor proteins. Within a recombinant expression vector, “operably linked” is intended to mean that the coding sequence of interest is linked to the regulatory sequence(s) in a manner that allows for expression of the coding sequence, e.g., in an in vitro transcription/translation system or in a host cell when the vector is introduced into the host cell.

“Vector” or “construct” refers to a nucleic acid molecule into which a heterologous or isolated nucleic acid can be or is inserted. A vector can be used to deliver the heterologous or isolated nucleic acid to the interior of a cell. Some vectors can be introduced into a host cell allowing for replication of the vector or for expression of a protein that is encoded by the vector or construct. Vectors typically have selectable markers, for example, genes that encode proteins allowing for drug resistance, origins of replication sequences, and multiple cloning sites that allow for insertion of a heterologous sequence. Numerous vectors are known in the art including, but not limited to, linear polynucleotides, polynucleotides associated with ionic or amphiphilic compounds, plasmids, and viruses. The properties, construction and use of such vectors, as well as other vectors, in the present invention will be readily apparent to those of skill from the present disclosure.

“Sequence” means the linear order in which monomers occur in a polymer, for example, the order of amino acids in a polypeptide or the order of nucleotides in a polynucleotide.

Unless defined otherwise, all technical and scientific terms used herein have the same meaning as commonly understood to one of ordinary skill in the art to which this invention pertains. In this application, certain terms shall have the meanings as set forth in the specification. It must be noted that as used herein and in the appended claims, the singular forms “a,” “an,” and “the” include plural referents unless the context clearly dictates otherwise.

According to embodiments of the present invention, several SNPs on human chromosome 4q25 are associated with an elevated risk of gout. Therefore, one aspect of the invention relates to a method of identifying a human subject having an elevated risk of gout and/or hyperuricemia by detecting the occurrence of at least one SNP associated with an elevated risk of gout in an ALPK1 gene in a biological sample from the human subject. The invention also relates to a method of identifying a human subject having an elevated risk of gout and/or hyperuricemia by determining the expression level of an ALPK1 gene in a biological sample from the human subject. The invention further relates to an isolated nucleic acid molecule, its complement or gene variant comprising at least one of the polymorphisms identified herein to be associated with gout and/or hyperuricemia.

The invention also relates to a kit for performing a diagnostic test to identify a human subject having an elevated risk of gout and/or hyperuricemia. The invention further relates to a method of selecting or identifying a compound useful for treating gout and/or hyperuricemia.

According to embodiments of the invention, twelve major SNPs each located in the ALPK1 gene have been found to be associated with an elevated risk of gout. The twelve major SNPs for risk assessment according to embodiments of the invention locate at specific nucleotide positions on SEQ ID NOs:1-12, respectively. The eleven major SNPs have been identified as risk alleles for gout based on their significantly higher odd ratios in gout cases than controls as determined by major alleles risk analysis.

As used herein, SEQ ID NOs:1-12 provide the plus strand sequences of the 12 SNPs according to embodiments of the invention. For each plus strand, there is a minus strand sequence, which is the exact complement to the plus strand. A plus strand and its corresponding minus strand may base pair with each other when in the anti-parallel orientation. The identity of the risk-associated nucleotide at a corresponding nucleotide position in the minus strand sequence of each of the SNPs of the invention, therefore is also predictive of risk of gout and/or hyperuricemia.

In one embodiment, a SNP associated with an elevated risk of gout comprises “C” at the nucleotide corresponding to nucleotide 15 of SEQ ID NO:1, or “G” at the nucleotide corresponding to nucleotide 15 of the corresponding minus strand of SEQ ID NO:1. As shown in Table 1, the SNP has the risk allele C, depicted in the online SNP database of the National Center for Biotechnology Information (NCBI), having an accession number (NCBI dbSNP ID) of rs916868.

In another embodiment, a SNP associated with an elevated risk of gout comprises “G” at the nucleotide corresponding to nucleotide 15 of SEQ ID NO:2, or “C” at the nucleotide corresponding to nucleotide 17 of the corresponding minus strand of SEQ ID NO:2. As shown in Table 1, the SNP has the risk allele G, depicted in NCBI dbSNP ID of rs9994944.

In another embodiment, a SNP associated with an elevated risk of gout comprises “A” at the nucleotide corresponding to nucleotide 15 of SEQ ID NO:3, or “T” at the nucleotide corresponding to nucleotide 16 of the corresponding minus strand of SEQ ID NO:3. As shown in Table 1, the SNP has the risk allele A, depicted in NCBI dbSNP ID of rs2074388.

In another embodiment, a SNP associated with an elevated risk of gout comprises “G” at the nucleotide corresponding to nucleotide 16 of SEQ ID NO:4, or “C” at the nucleotide corresponding to nucleotide 15 of the corresponding minus strand of SEQ ID NO:4. As shown in Table 1, the SNP has the risk allele G, depicted in NCBI dbSNP ID of rs13148353.

In another embodiment, a SNP associated with an elevated risk of gout comprises “A” at the nucleotide corresponding to nucleotide 15 of SEQ ID NO:5, or “T” at the nucleotide corresponding to nucleotide 16 of the corresponding minus strand of SEQ ID NO:5. As shown in Table 1, the SNP has the risk alleles A, depicted in NCBI dbSNP ID of rs2074379.

In another embodiment, a SNP associated with an elevated risk of gout comprises “C” at the nucleotide corresponding to nucleotide 15 of SEQ ID NO:6, or “G” at the nucleotide corresponding to nucleotide 17 of the corresponding minus strand of SEQ ID NO:6. As shown in Table 1, the SNP has the risk alleles C, depicted in NCBI dbSNP ID of rs11726117.

In another embodiment, a SNP associated with an elevated risk of gout comprises “C” at the nucleotide corresponding to nucleotide 16 of SEQ ID NO:7, or “G” at the nucleotide corresponding to nucleotide 15 of the corresponding minus strand of SEQ ID NO:7. As shown in Table 1, the SNP has the risk allele C, depicted in NCBI dbSNP ID of rs6841595.

In another embodiment, a SNP associated with an elevated risk of gout comprises “T” at the nucleotide corresponding to nucleotide 15 of SEQ ID NO:8, or “A” at the nucleotide corresponding to nucleotide 16 of the corresponding minus strand of SEQ ID NO:8. As shown in Table 1, the SNP has the risk allele T, depicted in NCBI dbSNP ID of rs11098156.

In another embodiment, a SNP associated with an elevated risk of gout comprises “G” at the nucleotide corresponding to nucleotide 15 of SEQ ID NO:9, or “C” at the nucleotide corresponding to nucleotide 16 of the corresponding minus strand of SEQ ID NO:9. As shown in Table 1, the SNP has the risk allele G, depicted in NCBI dbSNP ID of rs231247.

In another embodiment, a SNP associated with an elevated risk of gout comprises “A” at the nucleotide corresponding to nucleotide 16 of SEQ ID NO:10, or “T” at the nucleotide corresponding to nucleotide 15 of the corresponding minus strand of SEQ ID NO:10. As shown in Table 1, the SNP has the risk allele A, depicted in NCBI dbSNP ID of rs11098156.

In another embodiment, a SNP associated with an elevated risk of gout comprises “G” at the nucleotide corresponding to nucleotide 16 of SEQ ID NO:11, or “C” at the nucleotide corresponding to nucleotide 15 of the corresponding minus strand of SEQ ID NO:11. As shown in Table 1, the SNP has the risk allele G, depicted in NCBI dbSNP ID of rs231253.

In another embodiment, a SNP associated with an elevated risk of gout comprises “A” at the nucleotide corresponding to nucleotide 15 of SEQ ID NO:12, or “T” at the nucleotide corresponding to nucleotide 16 of the corresponding minus strand of SEQ ID NO:12. As shown in Table 1, the SNP has the risk allele A, depicted in NCBI dbSNP ID of rs960583.

It may be appreciated that some human subjects may have one or more polymorphisms in the context sequence according to embodiments of the invention. For instance, a human subject may have a polymorphism in the 3′ flanking sequence of the polymorphism at nucleotide 15 of SEQ ID NO:2 (rs9994944). And the methods of the invention may still be practiced with this human subject. In other words, the methods of the invention are not limited to those human subjects who have flanking sequences identical to the flanking sequences for any of the polymorphisms at specific nucleotide positions of SEQ ID NOs:1-12 described above.

According to some examples of the invention, the detection may also include detecting whether the human subject is heterozygous or homozygous for the polymorphism of interest. A person who is homozygous for a SNP allele associated with gout and/or hyperuricemia at any of the SNP positions according to embodiments of the invention may be at elevated risk of gout and/or hyperuricemia. A person who is heterozygous at any of the SNPs may also be at elevated risk of gout. However, a person who is homozygous at any of the SNPs may be at an increased risk compared to a person who is heterozygous.

In several examples of the invention, a target polynucleotide sequence may be isolated from a biological sample and amplified before detection of the SNP identity. The target polynucleotide sequence may be a region of the genomic DNA, mRNA or unprocessed RNA containing the SNP of interest. The genomic DNA, mRNA, and/or unprocessed RNA transcripts are isolated from the biological sample by conventional means known to the skilled artisan. The isolated genomic DNA, mRNA, and/or unprocessed RNA transcripts may be used, with or without amplification, to detect the SNP allele at one or more of the chromosome 4q25 SNPs shown herein to be associated with gout and or hyperuricemia. And the genomic DNA used for the diagnosis may be obtained from body cells, such as those present in peripheral blood, urine, saliva, bucca, surgical specimen, and autopsy specimens. In preferred embodiments, biological samples suitable for testing include blood, saliva, amniotic fluid, and tissue. The most preferred biological sample is blood. However, any biological sample from which genetic material or the products of the marker genes is isolated may also be practiced with the present invention.

Amplification of a target polynucleotide sequence may be carried out by any method known to the skilled artisan. Amplification methods include, but are not limited to polymerase chain reaction (PCR) including RT-PCR, strand displacement amplification, transcription base amplification, nucleic acid sequence-based amplification and repair chain reaction. In general, PCR involves treating a nucleic acid sample (e.g. in the presence of a heat stable DNA polymerase) with a pair of amplification primers. One primer of the pair hybridizes to one strand of a target polynucleotide sequence, particularly to nucleic acid containing the SNP to be detected, and not to nucleic acid that does not contain the SNP to be detected. The second primer of the pair hybridizes to the other, complementary strand of the target polynucleotide sequence, particularly to nucleic acid containing the SNP to be detected, and not to nucleic acid that does not contain the SNP to be detected. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained.

In some detection assays, a polynucleotide probe, which is used to detect DNA containing a SNP of interest, is a probe which binds to DNA encoding a specific SNP allele, but does not bind to DNA that does not encode that specific SNP allele under the same hybridization conditions. For instance, the detection probe may straddle a SNP site and discriminate between alleles. In other assays, the probe may bind to a sequence that is 3′ to the SNP and removed from the SNP by one or more bases. In some cases, the probe may be labeled with one or more labels.

The probes and primers are designed using the sequences flanking the SNP in the target polynucleotide sequence. SEQ ID NOs:1-12 provide flanking sequence information for each of the 12 SNPs according to embodiments of the invention. Depending on the particular SNP identification protocol utilized, the consecutive nucleotides of the region that hybridizes to a target polynucleotide sequence may include the target SNP position. Alternatively, the region of consecutive nucleotides may be complementary to a sequence in close enough proximity 5′ and/or 3′ to the SNP position to carry out the desired assay. The skilled artisan can readily design primer and probe sequences using the sequences provided herein in view of the present disclosure. Considerations for primer and probe design with regard to, for instance, melting temperature and avoidance or primer-dimers, are well known to the skilled artisan. A number of computer programs, such as Primer Express® and Primo SNP 3.4 can be readily used to obtain optimal primer/probe sets. The probes and primers may be chemically synthesized using commercially available reagents and synthesizers by methods well known in the art.

There are numerous methods of SNP identification known to the skilled artisan can be used to identify the SNPs of interest in view of the present disclosure. See, for instance, Kwok (2001, Annu. Rev. Genomics Hum. Genet. 2: 235-258) and Theohilus et al., (2002, PCR mutation Detection Protocols, Humana Press, Totowa, N.J.). SNP identification methods include, but are not limited to 5′ nuclease assay, primer extension or elongation assays, allele specific oligonucleotide ligation, allele specific hybridization, sequencing, invasive cleavage reaction, branch migration assay, single strand conformational polymorphism (SSCP), denaturing gradient gel electrophoresis (DGGE) and immunoassay.

The 5′ nuclease assay, also known as the 5′ nuclease PCR assay and the TaqMan™ assay (Applied Biosystems, Foster City, Calif.), provides a sensitive and rapid means of genotyping SNPs. The 5′ nuclease assay detects, by means of a probe, the accumulation of a specific amplified product during PCR. The probe is designed to straddle a target SNP position and hybridize to the target polynucleotide sequence containing the SNP position only if a particular SNP allele is present. During the PCR reaction, the DNA polymerase, which extends an amplification primer annealed to the same strand and upstream of the hybridized probe, uses its 5′ nuclease activity and cleaves the hybridized probe. There are different ways to detect the probe cleavage. In one common variation, the 5′ nuclease assay utilizes an oligonucleotide probe labeled with a fluorescent reporter dye at the 5′ end of the probe and a quencher dye at the 3′ end of the probe. The proximity of the quencher dye to the fluorescent reporter in the intact probe maintains a reduced fluorescence for the reporter. Cleavage of the probe separates the fluorescent reporter dye and the quencher dye, resulting in increased fluorescence of the reporter. The 5′ nuclease activity of DNA polymerase cleaves the probe between the reporter and the quencher only if the probe hybridizes to the target, and the target is amplified during PCR. Accumulation of a particular PCR product is thus detected directly by monitoring the increase in fluorescence of the reporter dye. In another variation, the oligonucleotide probe for each SNP allele has a unique fluorescent dye and detection is by means of fluorescence polarization.

The primer extension reaction involves designing and annealing a primer to a sequence downstream of a target SNP position in an amplified target polynucleotide sequence. A mix of dideoxynucleotide triphosphates (ddNTPs) and/or deoxynucleotide triphosphate (dNTPs) are added to a reaction mixture containing amplified target, primer, and DNA polymerase. Extension of the primer terminates at the first position in the PCR amplified target where a nucleotide complementary to one of the ddNTPs in the mix occurs. The primer can be annealed to a sequence either immediately 3′ to or several nucleotides removed from the SNP position. For single base or single nucleotide extension assays, the primer is annealed to a sequence immediately 3′ to the SNP position. If the primer anneals to a sequence several nucleotides removed from the target SNP, the only limitation is that the template sequence between the 3′ end of the primer and the SNP position can not contain a nucleotide of the same type as the one to be detected. Otherwise, this will cause premature termination of the extension primer. Alternatively, if all four ddNTPs alone, and no dNTPs, are added to the reaction mixture, the primer will always be extended by only one nucleotide, corresponding to the target SNP position. In this instance, primers are designed to bind to a sequence one nucleotide downstream from the SNP position. In other words, the nucleotide at the 3′ end of the primer hybridizes to the nucleotide immediately 3′ to the SNP position. Thus, the first nucleotide added to the primer is at the SNP. In one common variation, the ddNTPs used in the assay each has a unique fluorescent label, enabling the detection of the specific nucleotide added to the primer. SNaPshot™ from Applied Biosystems is a commercially available kit for single nucleotide primer extension using fluorescent ddNTPs, and can be multiplexed.

An alternative detection method uses mass spectrometry to detect the specific nucleotide added to the primer in a prime extension assay. For detection by mass spectrometry, extension by only one nucleotide is preferable, as it minimizes the overall mass of the extended primer, thereby increasing the resolution of mass differences between alternative SNP nucleotides. Furthermore, mass-tagged dideoxynucleoside triphosphate (ddNTPs) can be employed in the primer extension reaction in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer. The primers are extended, purified and then analyzed by MALDI-TOF mass spectrometry to determine the identity of the nucleotide present at the SNP position. MassARRAY™ (Sequenom, San Diego, Calif.) is a commercially available system for SNP identification using mass spectrometry.

Allele-specific oligonucleotide ligation uses a pair of oligonucleotide probes that hybridize to adjacent segments of sequence on a nucleic acid fragment containing the SNP. One of the probes has a SNP allele-specific base at its 3′ or 5′ end. The second probe hybridizes to sequence that is common to all SNP alleles. If the first probe has an allele-specific base at its 3′ end, the second probe hybridizes to sequence segment immediately 3′ to the SNP. If the first probe has an allele-specific base at its 5′ end, the second probe hybridizes to the sequence segment immediately 5′ to the SNP. The two probes can be ligated together only when both are hybridized to a DNA fragment containing the SNP allele for which the first probe is specific. One method of detecting the ligation product involves fluorescence. The second probe, which hybridizes to either allele, is fluorescently labeled. The allele-specific probe is labeled with biotin. Streptavidin capture of the allele-specific ligation product and subsequent fluorescent detection is used to determine which SNP is present. A commercially available kit, SNPlex™ (Applied Biosystems, Foster City, Calif.), may be used to analyze the ligation products.

Allele-specific hybridization distinguishes between two DNA molecules differing by one base using hybridization. Amplified DNA fragments containing the target SNP are hybridized to allele-specific oligonucleotides. As one variation, the amplified DNA fragments may be fluorescence labeled and the allele-specific oligonucleotides may be immobilized. In another variation, the allele-specific oligonucleotides are labeled with an antigen moiety. Binding may be detected via an enzyme-linked immunoassay and color reaction. In yet another variation, the allele-specific oligonucleotides are radioactively labeled.

The invasive cleavage assay uses two probes that have a one nucleotide overlap. When annealed to target DNA containing the SNP, the one nucleotide overlap forms a structure that is recognized by a 5′ nuclease that cleaves the downstream probe at the overlap nucleotide. The cleavage signal can be detected by various techniques, including fluorescence resonance energy transfer (FRET) on fluorescence polarization. Reaction conditions can be adjusted to amplify the cleavage signal, allowing the use of very small quantities of target DNA. Thus, the assay does not necessary require amplification of the target prior to detecting the SNP identity. The branch migration assay based on Holliday junction migration, involves the detection of a stable four-way complex for SNP identification (See, for instance, U.S. Pat. No. 6,878,530).

SNP can also be scored by direct DNA sequencing. A variety of automated sequencing procedures may be utilized when performing the diagnostic assays, including sequencing by mass spectrometry. Traditional sequencing methods may also be used, such as dideoxy-mediated chain termination method (Sanger et al., 1975, J. Molec. Biol. 94: 441; Prober et al. 1987, Science 238: 336-340) and the chemical degradation method (Maxam et al., 1977, PNAS 74: 560). A preferred sequencing method for SNPs is pyrosequencing.

Pyrosequencing involves a cascade of four enzymatic reactions that permit the indirect luciferase-based detection of the pyrophosphate released when DNA polymerase incorporates a dNTP into a template-directed growing oligonucleotide. Each dNTP is added individually and sequentially to the same reaction mixture, and subjected to the four enzymatic reactions. Light is emitted only when a dNTP is incorporated, thus signaling which dNTP is incorporated. Unincorporated dNTPs are degraded by apyrase prior to the addition of the next dNTP. The method can detect heterozygous individuals in addition to heterozygotes. Pyrosequencing uses a single stranded template, typically generated by PCR amplification of the target sequence. One of the two amplification primers is biotinylated thereby enabling streptavidin capture of the amplified duplex target. The captured duplex is denatured by alkaline treatment, thereby releasing the non-biotinylated strand. The detection primer used for SNP identification using pyrosequencing is designed to hybridize to a sequence 3′ to the SNP. In a preferred embodiment, the 3′ sequence is immediately adjacent to the SNP position. Thus, the SNP identity is ascertained when the first nucleotide is incorporated.

Further examples of methods used to identify for the SNPs of the present invention include single-strand conformational polymorphism (SSCP) and denaturing gradient gel electrophoresis (DGGE). SSCP identifies base differences by alteration in electrophoretic migration of single stranded PCR products, as described in Orita et al., (1989, PNAS 86: 2766-2770). Single-stranded PCR products can be generated by heating or otherwise denaturing double-stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel. And immunoassay methods using antibodies specific for SNP alleles can also be used for SNP detection.

In accordance with the present invention, the association between an ALPK1 polymorphism and gout and or hyperuricemia is typically identified by statistical analysis of the population data. As will be recognized by those skilled in the art, many known statistical methods, statistical tools and models can be used for this analysis. And an association is identified when the result of statistical analysis shows an association between an ALPK1 polymorphism and gout susceptibility, with at least 80%, 85%, 90%, 95%, or 99%, preferably 95% confidence.

A method according to an embodiment of the invention comprises detecting the presence of two or more SNPs associated with gout and/or hyperuricemia in an ALPK1 gene in a biological sample of a human subject. In an embodiment of the present invention, the two or more SNPs are detected using a collection of nucleic acid molecules. The collection of nucleic acid molecules can be a multi-well microassay plate or a microarray.

Studies have shown alcohol intake contributes to overproduction of urate by net ATP degradation to AMP (Yamamoto et al., Clin Chim Acta 356: 35-57 (2005)). Alcohol intake also increases the production of lactate which is a substrate for an urate transporter, URAT1 responsible for urate reabsorption or competitive inhibition of renal urate secretion. An overall effect of alcohol intake may result in both overproduction and reduced renal excretion of urate, leading to high serum urate level in serum urate disorders, including but not limited to hyperuricemia and gout. Accordingly, the identification method according to an embodiment of this invention may also include determining the alcohol consumption habit and fractional excretion of uric acid (FEUA) of a human subject, in addition to the detection of the SNPs described above. In accordance with one embodiment of the invention, statistical analyses and models may be adopted to estimate with a statistical significance the risk size of the risk alleles and alcohol consumption as an adjusted confounding factor in the statistical model. As a specific embodiment of the invention, univariate analysis is conducted to show significant difference in habitual alcohol consumption between gout and nongout groups. And multiple logistic models may be used to estimate with at least 95% confidence intervals the odd ratios for heterozygotes and homozygotes of ALPK1 alleles when alcohol consumption habit is included as a covariate. In another embodiment of the invention, the joint effect of ALPK1 genotypes and alcohol consumption on gout risk are evaluated on both multiplicative and additive scales. A likelihood ratio test is used to test interaction between genetic traits and environmental factors based on a multiplicative model. In once other embodiment, the association in the ALPK1 polymorphism and parameters of uric acid levels and FEUA percentage are assessed using a mixed linear model analyses from statistical tool such as SAS software.

Similarly, the identification method may also include determining one or more other factors known to raise an individual's risk of gout, such as high purine diet, age and gender.

It is further discovered, for the first time, that the ALPK1 gene expression level is significantly higher in patients with gout and/or hyperuricemia than those in controls. Accordingly, the invention also relates to a method of identifying a human subject having an elevated risk of gout and/or hyperuricemia by determining the expression level of an ALPK1 gene in a biological sample from the human subject, wherein an elevated expression level of the ALPK1 gene is indicative of an elevated risk of gout and/or hyperuricemia in the human subject.

The expression level of an ALPK1 gene may be determined using methods well known to the skilled artisans. As one example of the invention, the expression level of the ALPK1 gene may be determined by reverse transcription polymerase chain reaction (RT-PCR), such as quantitative real-time RT-PCR (qRT-PCR) using a pair of primers that hybridize to an ALPK1 transcript under stringent hybridization conditions. The gene expression can also be detected by other molecular analysis such as Northern blot analysis. The expression level of an ALPK1 gene can also be analyzed indirectly by measuring the level of ALPK1 protein in a biological sample using known methods, such as Western blot analysis or ELISA. The expression level of an ALPK1 gene can also be analyzed indirectly by measuring functional role of ALPK1 gene in the development of gout and/or hyperuricemia, such as by measuring the biological activity, e.g., protein kinase activity, of the ALPK1 protein in a biological sample using methods known in the art. An elevated expression level of the ALPK1 gene may be indicative of an elevated risk of gout and/or hyperuricemia in the human subject.

The ALPK1 protein is a serine/threonine-protein kinase and highly expressed in the liver and kidney. It is a novel class of protein kinase with catalytic domain adapted to recognizing and phosphorylating amino acid residues surrounded by peptides have an α-helical conformation via ATP-triggered catalytic reactions. ALPK1 protein contains a glycine-rich motif typical of ATP-binding sites in the catalytic domain of conventional protein kinases.

Several organic anion transporters (OATs), which contain 12 membrane-spanning α-helices, can transport urate (Eraly et al., Mol Pharmacol 65: 479-487 (2004); Anzai et al., Curr Opin Rheumatol 19: 151-157 (2007)). For example, both OAT1 and OAT3 proteins are responsible for mediating urate secretion (Bakhiya et al., Cell Physiol Biochem 13: 249-256 (2003)). Studies showed that OATs may be phosphorylated by protein kinases to inhibit their activity on urate secretion (Hediger et al., Physiology (Bethesda) 20: 125-133 (2005)). The cytoplasmic domains of OATs contain PDZ motifs responsible for protein targeting and protein complex assembly and phosphorylation sites for protein kinase A and protein kinase C.

Because ALPK1 has a catalytic domain adapted to recognizing and phosphorylating amino acids surrounding α-helices, ALPK1 may phosphorylate OATs thereby downregulating the activity of OATs and increasing excretion of uric acid, thus resulting in treatment of the symptoms of gout or hyperuricemia.

Accordingly, the present invention further relates to methods of selecting a compound useful for treating gout and/or hyperuricemia. The compound may directly or indirectly inhibit phosphorylation of OAT by the ALPK1 protein. The compound may also directly or indirectly decrease the expression of ALPK1 gene.

The compound identification methods can be performed using conventional laboratory formats or in assays adapted for high throughput. The term “high throughput” refers to an assay design that allows easy screening of multiple samples simultaneously and/or in rapid succession, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid handling experiments. It will be appreciated by those in the art that as miniaturization of plastic molds and liquid handling devices are advanced, or as improved assay devices are designed, greater numbers of samples can be processed using the design of the present invention, in view of the present disclosure.

Candidate compounds encompass numerous chemical classes, including but not limited to, small organic or inorganic compounds, natural or synthetic molecules, such as antibodies, proteins or fragments thereof, antisense nucleotides, interfering RNA (RNAi) and ribozymes. Preferably, they are small organic compounds, i.e., those having a molecular weight of more than 50 yet less than about 2500. Candidate compounds comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate compounds can comprise cyclic carbon or heterocyclic structure and/or aromatic or polyaromatic structures substituted with one or more of the above-identified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound typically is a DNA or RNA molecule, although modified nucleic acids having non-natural bonds or subunits are also contemplated.

Candidate compounds are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Candidate compounds, based on the present disclosure, can also be obtained using any of the numerous approaches in combinatorial library methods known in the art, including biological libraries; spatially addressable parallel solid phase or solution phase libraries: synthetic library methods requiring deconvolution; the “one-bead one-compound” library method; and synthetic library methods using affinity chromatography selection (Lam (1997) Anticancer Drug Des. 12:145). Alternatively, libraries of natural compounds in the form of bacterial, fungal, plant and animal extracts are available or readily produced. Additionally, natural and synthetically produced libraries and compounds can be readily modified through conventional chemical, physical, and biochemical means. Examples of methods for the synthesis of molecular libraries can be found in the art, for example in: Zuckermann et al. (1994). J Med. Chem. 37:2678. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,571,698), plasmids (Cull et al. (1992) Proc. Natl. Acad. Sci. USA 89:1865-1869) or phage (see e.g., Scott and Smith (1990) Science 249:3 86-390).

In one embodiment of the present invention, the compound identification method involves identifying a compound that directly or indirectly inhibits phosphorylation of an OAT1 or OAT3 protein by an ALPK1 protein. Accordingly, a test compound in a buffering solution is contacted with a first polypeptide comprising a catalytic domain of an ALPK1 protein and a second polypeptide comprising a phosphorylation site for the ALPK1 protein in an OAT1 or OAT3 protein. When the test compound is contacted with the first and second polypeptides, a change in phosphorylation level of the second polypeptide is detected. The test compound is then selected based on its ability to decrease the phosphorylation level as compared to a control measurement wherein only the buffering solution, and not the test compound, is contacted with the first and second polypeptides.

A variety of other reagents can be included in the mixture. These include reagents such as salts, buffers, neutral proteins (e.g., albumin), detergents, etc. that can be used to facilitate optimal protein-protein binding. Such a reagent can also reduce non-specific or background interactions of the reaction components. Other reagents that improve the efficiency of the assay such as protease inhibitors, antimicrobial agents, and the like can also be used.

As examples of the invention, the first polypeptide can be isolated or purified, or present in a cell lysate. In a particular embodiment, the first polypeptide is a product of expression of a recombinant host cell for the first polypeptide. The recombinant host cell comprises a recombinant polynucleotide encoding an ALPK1 protein catalytic domain. The first polypeptide can be, for example, a human ALPK1 protein catalytic domain; a full length human ALPK1 having an amino acid sequence of SEQ ID NO:13; or a fusion protein having a protein tag fused to a human ALPK1 protein catalytic domain. The protein tag facilitates purification or isolation of the fusion protein, as would be known to the art in view of the present disclosure.

As examples of the invention, the second polypeptide may be a product of expression of a recombinant host cell for the second polypeptide. The recombinant host cell may comprise a recombinant polynucleotide encoding the phosphorylation sites for an ALPK1 protein on an OAT1 or OAT3 protein. The recombinant host cell may comprise a recombinant polynucleotide encoding the human OAT1 or OAT3 cytoplasmic domain. The second polypeptide can be, for example, a human OAT1 or OAT3 protein cytoplasmic domain; a full length human OAT1 or OAT3 having an amino acid sequence of SEQ ID NO:14 or SEQ ID NO:15, respectively; or a fusion protein having a protein tag fused to a human OAT1 or OAT3 protein cytoplasmic domain. The protein tag facilitates purification or isolation of the fusion protein. The second polypeptide can be present in a cell lysate. In some examples of the invention, the second polypeptide may be associated with an isolated membrane. As a further example, the second polypeptide may be present on the surface of a cell.

In another embodiment of the present invention, the compound identification method involves identifying a compound that directly or indirectly decreases the expression of an ALPK1 gene. The method includes contacting a test compound with a host cell that expresses a gene operably linked to a regulatory sequence for an ALPK1 gene; detecting a change in the expression level of the gene from the host cell; and selecting the test compound by its ability to decrease the expression level of the gene, as compared to a control measurement which is taken when only the buffering solution, not the test compound, is contacted with the host cell.

The mechanism for regulating the expression of an ALPK1 gene includes the mechanism by which nuclear, cytoplasmic, or intracellular factors influence the control of gene action at the level of transcription or translation. For example, the mechanism includes gene activation or gene repression. The cell comprising a mechanism for regulating the expression of an ALPK1 gene can be a native host cell that expresses an ALPK1 endogenously. The cell can also be a recombinant cell containing a recombinant DNA sequence having a regulatory sequence for an ALPK1 gene, and the regulatory sequence is operably linked to a gene, preferably a reporter gene.

The effect of the compound on the expression of a gene controlled by the regulatory sequence of an ALPK1 gene can be measured in a variety of ways. For example, the effect can be measured by the amount of mRNA or protein of the gene from the cell, or by the activity of the gene product from the cell. When a reporter gene is used, the effect can be measured as the level of reporter gene product from the cell. For example, when the ALPK1 regulatory sequence is operably linked to a GFP gene, the effect of the compound on gene expression can be measured as the effect of the compound on emissions of green fluorescence from the cell using a fluorometer. When an endogenous ALPK1 cell is used, the effect of the compound on gene expression can be measured by the amount of ALPK1 mRNA or protein inside the cell using methods such as Northern Blot, qRT-PCR, SDS-PAGE, Western Blot, immunohisto- or immunocytochemistry, radioreceptor ligand binding, etc. Alternatively, the kinase activity of ALPK1 can be measured to evaluate the effect of the compound on the expression of the ALPK1 gene.

The cell-based method described herein not only identifies compounds that regulate ALPK1 gene expression directly via binding to one or more than one regulatory sequence of the ALPK1 gene, but also identifies compounds that regulate ALPK1 expression indirectly via binding to other cellular components whose activities influence ALPK1 expression or protein stability. For example, compounds that regulate the activity of a transcriptional activator or inhibitor for ALPK1 genes can be identified using the method described herein. Compounds that regulate the activity of a protease that degrades the ALPK1 protein in vivo can also be identified.

In another embodiment of the present invention, the method of compound identification further comprises administering the test compound to an animal to determine the effect of the test compound on a symptom related to gout and/or hyperuricemia in the animal. Examples of the symptoms related to gout and/or hyperuricemia in the animal include, pain, swelling, redness, warmness and stiffness in the joint. Gout usually attacks the big toe, however it can also affect other joints such as the ankle, heel, instep, knee, wrist, elbow, fingers, and spine. As a specific example, the effect of the test compound may be determined by observing a change of serum urate level in the gout subject after the test compound is administered to the subject for a period of time. In one specific example, uric acid levels and fractional excretion of uric acid may be determined from the gout subject before and after the test compound is administered.

The test compound may be prepared and administered in various formulations, preferably in liquid formulations, liquid suspensions or in the form of a dry product intended for reconstitution with water or isotonic saline, to the animals which include but are not limited to humans, other primates and mammals with hyperuricemia and/or gout. Other possible formulations, such as nanoparticles, liposomes, resealed erythrocytes and immunologically based systems may also be used to administer the test compound according to the method of the inventions. In accordance with another example of the invention, the effect of the test compound is then determined by observing an increased renal urate secretion of the animal as compared to a control measurement of the urate secretion taken when only the buffering solution is administered to the animal.

The invention also relates to kits, which can be used, for example to perform methods of the invention. Thus, the invention provides a kit for performing a diagnosis test to identify a human subject having an elevated risk of gout and/or hyperuricemia. The kit may include an isolated primer, primer pair, probe, or other specific binding pair member of the present invention, or a combination thereof designed for polymerase chain reaction amplification of a target polynucleotide sequence, which includes a SNP in an ALPK1 gene in a biological sample from a human subject. The kit may further include instructions and reagents for amplifying the target polynucleotide using a primer pair. The reagents may include at least one detectable label, which can be used to label the isolated oligonucleotide probe, primer, primer pair, or other specific binding pair member, or can be incorporated into a product generated using the isolated oligonucleotide probe, primer, primer pair, or specific binding pair member. The kit may also include at least one polymerase, ligase, or endonuclease, or a combination thereof. The kit may further include at least one polynucleotide corresponding to a portion of an ALPK1 gene comprising a SNP associated with increased risk of gout and/or hyperuricemia according to embodiments of the invention.

In addition, a kit of the invention may contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedures, or other enzyme or enzymes, which can be useful for performing am oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody.

In one embodiment, a kit of the invention includes one or more primer pairs of the invention, such as a kit being useful for performing an amplification reaction, e.g., a PCR amplification. Such a kit also can contain, for example, one or more reagents for amplifying a polynucleotide using a primer pair of the kit. The primer pair(s) can be selected, for example, such that they can be used to determine the SNP occurrence of a polynucleotide of a sample, at a position corresponding to a specified nucleotide of SEQ ID NOs:1-12. For example, the pair of primers can have a forward primer that selectively hybridizes to a sequence of the target polynucleotide upstream of a SNP position on one strand, and a reverse primer that selectively hybridizes to a sequence of the target polynucleotide upstream of the SNP position on a complementary strand. Therefore, an amplified product resulted from the amplification reaction may include the SNP loci.

In another embodiment, a kit of the invention provides a plurality of oligonucleotides of the invention, including one or more oligonucleotide probes or one or more primers, including forward and/or reverse primers, or a combination of such probes and primers or primer pairs. Such a kit provides a convenient source for selecting probe(s) and/or primer(s) for identifying one or more SNPs or haplotype alleles as desired. Such a kit also can contain probes and/or primers that conveniently allow a method of the invention to be performed for genotyping an individual's biological sample.

The invention also relates to an isolated nucleic acid molecule comprising a SNP of an ALPK1 gene selected from a group consisting of polymorphisms corresponding to specific nucleotide positions of SEQ ID NOs:1-12. The isolated nucleic acid molecules may be RNA, mRNA, DNA, cDNA, and may be double- or single-stranded. They may encode the sense or plus strand, the non-coding regions, or the antisense or minus strand. The nucleic acid molecule can include all or a portion of the coding sequence of the gene and can further comprise additional non-coding regions, such as introns and non-coding 3′ and 5′ sequences including regulatory sequences for example.

An embodiment of the present invention relates to a collection that comprises at least two of the isolated nucleic acid molecules according to embodiments of the invention. The nucleic acid molecules of the collection can be affixed to a solid surface in an array, present in separate wells on a plate, etc.

Also described herein are systems of vectors and host cells that include an isolated nucleic acid molecule having a SNP of an ALPK1 gene. A variety of expression vectors may be used in the present invention which include, but are not limited to, plasmids cosmids, phage, phagemids, or modified viruses. Typically, such expression vectors comprise a functional origin of replication for propagation of the vector in an appropriate host cell, one or more restriction endonuclease sites for insertion of the ALPK1 variant gene sequence, and one or more selection markers. The expression vector must be used with a compatible host cell which may be derived from a prokaryotic or an eukaryotic organism including but not limited to bacteria, yeasts, insects, mammals, and humans.

The expression constructs and vectors may be introduced into host cells for the purpose of producing the ALPK1 variant encoded by the ALPK1 gene containing SNPs associated with gout and/or hyperuricemia. The expression vector described herein may be synthesized and assembled from known DNA sequences by well known techniques in the art. The regulatory regions and enhancer elements can be of a variety of origins, both natural and synthetic. In accordance with examples of the invention, the host cell may include a gene operably linked to a regulatory sequence for ALPK1 gene. Specifically, the gene may be a reporter gene which encodes a protein selected from the group consisting of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase. Any cell type that can produce mammalian protein and is compatible with the expression vector may be used, including those that have been cultured in vitro or genetically engineered. Host cells may be obtained from normal or affected subjects, including healthy humans, private laboratory deposits, public culture collections such as American Type Culture Collection, or from commercial suppliers. Moreover, vectors and host cells may also be obtained commercially.

This invention will be better understood by reference to the specific, non-limiting examples that follow, but those skilled in the art will readily appreciate that the examples are only illustrative of the invention as described more fully in the claims which follow thereafter.

### EXAMPLE 1

### Study Subjects

A population-based epidemiology survey in Dayan tribe communities in Taiwan was conducted during 2004-2007. All 1522 subjects including 917 male (422 gout cases and 495 controls) and female 605 (126 gout cases and 479 controls), responded to questionnaires on the information, such as demographic information (age, gender, and ethnicity), alcohol consumption (the frequency of intake of alcoholic beverages), general and detailed medical history including current/past medications, during the survey conducted at the local health stations in Taiwan, with the aim of health education and disease prevention.

The diagnosis of gout was confirmed by a rheumatologist based on the preliminary criteria for the classification of the acute arthritis of primary gout (Wallace et al., Arthritis Rheum 20: 895-900 (1977)). The study was approved by the Human Ethical Committee of Kaohsiung Medical University and National Health Research Institutes in Taiwan. Blood was drawn by trained medical technologists. Routine blood tests were conducted using automated analyzer (Beckman LX-20, Palo Alto, Calif.) to measure biochemical indexes, such as plasma total cholesterol, triglyceride, creatine and uric acid for the study subjects. Total genomic DNA was obtained from white blood cells using a genomic DNA extraction kit (PureGene DNA Purification Kit) (Gentra Systems, Minneapolis, Minn.) and stored at −20° C. until genotyping. Informed consent for collecting blood samples, anthropometric measurements and the survey questionnaires was obtained for each study subject before the study.

### EXAMPLE 2

### Identification of ALPK1 Alleles

A linkage analysis with fine mapping was conducted to locate the 4q25 candidate region at 114 cM-124 cM of the human chromosome 4. Five microsatellite or short tandem repeat polymorphism (STRP) markers, including AFMa302wb5, AFM164tf6, AFM186×4, AFM319yg9 and AFM151×c3 listed in Table 1 were introduced based on the 21 gouty families with identified linkage using CEQ™ 8000 Genetic Analysis System (Beckman Coulter, Fullerton, Calif.).

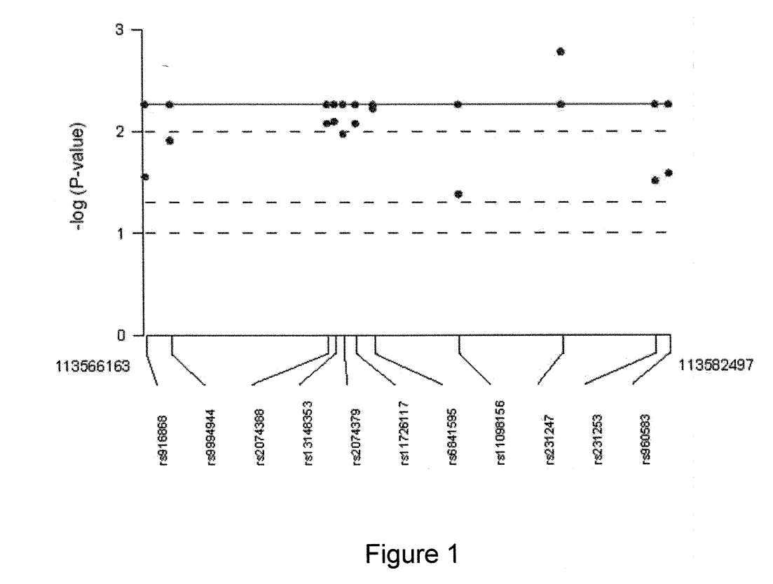

Next, a case-control study was conducted with at least 666 polymerase-chain-reaction (PCR) amplicons of thirty-eight genes located between D4S1647 and D4S2937 using a gene-centric approach with gout risk. The resequencing panel consisted of 23 unrelated paired gout cases and controls in males from Dayan community with at least 404 SNPs being typed and discovered. Eight SNPs had call rate <90% and were then excluded. Then from the next association study, four suggestive candidate genes (SCYE1, DKK2, FLJ39370 and ALPK1 genes) were determined for set of variants that were enriched or depleted in gout cases. The significantly high-priority position comprising 75 SNPs was validated and/or identified and examined in the family and population based set of 201 gout cases and 244 controls. ALPK1 gene was found to be significantly associated. The analysis was further narrowed down to assess the association between these 12 SNPs of ALPK1 gene (4 missense: rs2074388 G565D, rs13148353H642R, rs2074379 M7321 and rs11726117 M861T, located in exon 11; 2 nonsense: rs231247 R1084R, located in exon 13; rs55840220 T1145T, located in exon 14; rs916868, located in intron 6; rs9994944, located in intron 7; rs6841595, located in intron 11; rs11098156, located in intron 12; rs231253 and rs960583, both located in 3′-upstream regulatory region) with risk of gout in additional 271 subjects (134 cases and 137 controls). Subsequently, examination of the allele frequencies indicates that pairs of 12 SNPs were likely to be in complete linkage disequilibrium with each other (disequilibrium coefficient [D′]≧0.80) and spanned 16.3 kb in 335 gout cases and 381 controls. Permutation of global haplotype showed a highly significantly finding among 11 SNPs of ALPK1 (p=0.005) as shown in FIG. 1. We then tested remaining 8 SNPs, totaling 1522 subjects (548 gout cases and 974 controls) were entered into the correlation analysis.

The multipoint analysis was conducted using the conditional-logistic model (Olson (1999) Am. J. Hum. Genet. 65: 1760-1769) implemented in the S.A.G.E. program package (version 5.4.1) to analyze the linkage. Linkage was analyzed by calculation of LOD (log of odds) values.

The one-parameter model was used along with the default value that constrained the relative risks λ₂=3.63λ₁−2.364, assuming that the expression fix for the mode of inheritance at a value approximately halfway between a dominant and a recessive model and correspond to the Whittemore and Tu minmax model of inheritance parameter (Whittemore and Tu (1998) Am. J. Hum. Genet. 62: 1228-1242). Likelihood-ratio statistics (LRS) was computed by multiplying the LOD score by 4.6.

The protocol for high throughput DNA resequencing has been established in the SNP discovery. All SNPs for the associated studies were detected by PCR-based bi-directional sequencing. PCR reactions were performed in a volume of 10 μl containing genomic DNA (10 ng), forward and reverse primers (0.5 μM each), dNTP (0.2 mM), 0.02 U Takara Ex Taq and 1×PCR buffer (Takara Biotechnology, Tokyo, Japan). The PCR amplification was initiated by a thermal cycler (GeneAmp PCR System 9700, Applied Biosystems) using the specific temperature cycling profile below:

| | |
|---|---|
| Pre-reaction incubation: | 95° C. for 5 minutes |
| 45 cycles | |
| denature: | 98° C. for 15 seconds |
| anneal: | 60° C. or 62° C. or 65° C. for 30 seconds |
| extend: | 72° C. for 1 minute |
| final extension: | 72° C. for 2 minutes |

Oligonucleotide primers were designed with the use of Primer3 software available online (Rozen et al., Methods Mol Biol 132: 365-386, 2000).

PCR products were treated with exonuclease I and shrimp alkaline phosphatase (USB Corporation, Cleveland, Ohio) to remove excess primers. The PCR products were directly sequenced on both strands with the same primers used in the PCR amplification. The sequencing reaction was performed using the BigDye Terminator v3.1 Cycle Sequencing Kit (Applied Biosystems) and analyzed on ABI 3730×1 DNA analyzer (Applied Biosystems). The sequences were aligned to detect SNP by using PolyPhred/Phred/Phrap/Consed with version 5.04/0.020425.c/0.990329/15 (www.phrap.org). The entire ALPK1 gene was screened for polymorphisms, and 44 SNPs were identified.

In stage I, multipoint analysis results showed peak strength linkage signal moving from 114 cM to 117 cM with a LOD score of 4.46 (p=3×10⁻⁵) when STRP marker such as AFM 164tf6 at 117 cM was added for fine-mapping the gout susceptibility gene locus at chromosome 4q25. When the alcohol consumption was included as a covariate in the model, the LOD score was significantly increased to 6.08 (p=1.84×10⁻⁵). In stage 1V, the SNP (SNP ID rs231247) of ALPK1 gene was found to have a linkage with the LOD score of 5.34, and the LOD score was increased to 6.29 after the alcohol consumption was included as the covariate. In addition, the LOD score was increased to 6.05 (p=2×10⁻⁶) when other SNPs, such as two major SNPs (rs9994944 and rs13148353) located on the ALPK1 gene were also included in the conditional logistic model.

### EXAMPLE 3

### Association of ALPK1 SNPs with Gout in Taiwanese Aborigines

In the association study, data was analyzed using the Statistical Analysis Systems (SAS) software version 9.1.3 (SAS institute Inc, North Carolina), SAS/GENETIC, Haploview 4.0 and PLINK v1.00. Possible confounding bias arising from family effects and population substructure were analyzed using a GLIMMIX procedure provided by SAS software. The variance of the random family effects was rather small (rs231241, estimate=0.39, 95% confidence interval [CI]-0.68-1.44). The variance of the random tribe's effects was also rather small (rs231247, estimate=0.61, 95% CI-1.09-2.31). Differences between the gout and non gout groups were assessed by the Chi square (X²) test for categorical variables. Odd ratios were calculated with 95% confidence intervals to estimate the risk size for the heterozygotes and homozygotes for risk alleles, and adjusted confounding factors including age, gender, tribes, familial aggregation of gout and alcohol drinking in multiple logistic regression models.

One SNP (rs231247 of ALPK1) for which there was case-control study evidence for main effects with gout risk was identified. Several SNPs within, and in proximity to, the ALPK1 gene showed strong associations with gout risk were listed in Table 2. The genotypic and allelic p-value showed significant risk of gout for different subgroups (p<0.05). In a combined analysis comprising 548 gout cases against 974 controls, SNP rs231247 was the most strongly associated with gout. From Table 3, homozygosity for allele (G) of rs231247 was found to associate with a significant risk of gout (odds ratio [OR] 1.80, 95% CI 1.32-2.46, p=4.60×10⁻⁴), whereas the allelic odds ratio was 1.36 (95% CI 1.17-1.59, p=5.00×10⁻⁵, G allele: 62% in gout cases versus 54% in controls). And since gout was characterized by elevated serum urate, investigation for the correlation between the ALPK1 gene and hyperuricemia was also conducted. Results of association of 12 ALPK1 gene SNPs in hyperuricemia cases and controls were shown in Table 4.

SNPs having a minor allele frequency of less or equal to 5% or a p-value of less or equal to 0.05 from Hardy-Weinberg equilibrium test were excluded. There were 12 SNPs in the ALPK1 gene with significantly higher odd ratios in gout cases than controls as determined by major alleles risk analysis. The most significant risk of gout and/or hyperuricemia was associated with the SNP of SNP ID: rs231247. The G/G homozygote for rs231247 has an odd ratio ranging from 1.32 to 2.46, preferably 1.80 with a 95% confidence interval (CI). The odd ratio of the G/G homozygote for rs231247 was increased to 1.98 to 3.46, preferably 2.62 with a 95% CI after being adjusted with the covariates in multiple logistic regression models.

### EXAMPLE 4

### Gene-Environment Analysis

The gene environment interaction analyses were used for unexposed alcohol consumption with no risk of genotype or allele as the reference group. Odd ratios were also computed for interactive effects of alcohol consumption and ALPK1 genotype or alleles susceptible for gout and/or hyperuricemia in all other groups using multiple logistic regression models.

The joint effects of ALPK1 genotypes and alcohol consumption on gout and/or hyperuricemia risk were evaluated on both multiplicative and additive scales. Age, gender, familial aggregation and alcohol consumption were associated with gout in univariate analysis. Multiple logistic models showed statistical significance after adjusting for covariates indicated alcohol consumption as independent effects. The joint risk between alcohol consumption and carrier of the gout-risk allele of rs231247 was 4.30-fold (2.30-8.04) with 95% CI for genotype GG (Table 5) and 3.31-fold (2.45-4.47) with 95% CI for G allele (Table 6).

Evidence of synergy was found for the joint risk of gout for ALKP1 genotypes (rs231247) and alcohol consumption as shown in Table 5. By comparison with non-drinkers possessing homozygous AA allele of rs231247, drinkers with AA genotype and non-drinkers with GG genotype had a 2.45 and 1.35 fold higher risk, respectively. The observed joint risk (adjusted odd ratio of 4.30) for drinkers possessing homozygous GG genotype of rs231247 was significantly higher than the non-drinkers possessing homozygous AA genotype. Drinkers with G allele of rs231247 had a supra-multiplicative combined risk of gout (adjusted odd ratio of 3.31). Further, rs231247 at-risk GG genotype and alcohol consumption accounted for 30.5% of the gout and/or hyperuricemia cases. Overall, most of the gout and/or hyperuricemia cases were attributable to the genetic trait and environmental factor.

### EXAMPLE 5

### Pairwise Standard Linkage Disequilibrium (LD) and Haplotype Estimate Analysis

Haplotype estimate analysis was performed by applying the R-based algorithm in snp.plotter plotting package program that conveyed both SNP/haplotype association and LD plotting information in a single graphic illustration for genetic analysis.

Pairwise global haplotype and linkage disequilibrium were estimated, and plotted according to their genomic positions as illustrated in FIG. 1. To locate susceptibility alleles, LD around ALPK1 was estimated using genotyping data for case samples. The pairwise LD structure was constructed using the software Haploview, version 4.0. Elevent SNPs spanning a 16.7 kb region of ALPK1 gene were selected based on at-risk major allele frequencies. Pairwise LD between polymorphisms was expressed in a normalized measure (D′) for assessing the strength of LD. The four missense gout-associated SNPs of ALPK1 had a strong disequilibrium coefficient (r²≧0.88). Within this block, a haplotype association analysis between at-risk haplotype (AGCG) (rs2074388 A>G, rs13148353 G>A, rs1726117 C>T and rs231247 G>A) and gout risk was performed. From Table 7, the at risk haplotype (AGCG) remained significant after 100,000 permutation analysis (p=8×10⁻⁴) and the odds ratio for the AGCG haplotype (frequency, 0.56 among cases and 0.49 among controls subject) was 1.29 (1.10 to 1.51) with 95% CI per copy of the haplotype, as compared with GATA haplotype (frequency, 0.35 among cases and 0.39 among controls subject). Therefore, it was evident that a haplotype association one variation at ALPK1 was sufficient to contribute to gout risk.

In this study, all haplotype pairs were in strong linkage disequilibrium in both gout (D′≧0.88) and control (D′≧0.81) groups. The global haplotype based on the 11 SNPs was found to be significantly associated with gout subjects (p<0.05) as shown by a solid line connecting 11 SNPs in FIG. 1.

### EXAMPLE 6

### ALPK1 mRNA Expression

Quantitative Real-Time Reverse Transcriptase Polymerase Chain Reaction (qRT-PCR)

The total RNA was isolated from human blood using PAXgene Blood RNA Kit (Qiagen) and reverse transcribed by TaqMan RT-PCR using reverse transcription reagents (Applied Biosystems). The reverse transcription reaction was set up in a 10 μl volume containing 2 μg total RNA, 2×RT buffer, 8 mM dNTP mixture, 2× random primers, 0.1× MultiScribe™ Reverse Transcriptase and RNase-free water. The reagents used were from an Advantage® RT-for-PCR Kit (Catalog Number 639506, Clontech, Takara Bio Company). The reverse transcription was performed with ABI 9700 with the following conditions: 25° C. for 10 min, 37° C. for 120 min, 85° C. for 5 min and cooled at 4° C. to generate cDNA encoding for ALPK1.

The TaqMan RT-PCR was carried out with pre-designed gene-specific TaqMan probes and primer pairs at the following specific temperature cycling profile of a real time thermal cycler system (Applied Biosystems 7900HT Fast Real-Time PCR System). Each amplification reaction contained 50 ng cDNA templates obtained from the reverse transcription reaction described above, 1× probe, 1× primers, and 10 μl TaqMan Universal PCR Master Mix. Final reaction volumes were 20 μl each.

| | |
|---|---|
| Pre-reaction incubation: | 50° C. for 2 minutes |
| denature: | 95° C. for 10 minutes |
| 40 cycles: | |
| amplify: | 95° C. for 15 seconds |
| anneal and extend: | 60° C. for 60 seconds |

The pre-designed gene-specific TaqMan probe and the pair of primers used in the TaqMan RT-PCR reaction were purchased from Applied Biosystems, as the Inventoried and Made to Order Assay with Assay ID No: Hs00228473_ml.

Each expression experiment was carried out in triplicates reactions per sample, and a control without template was included in each plate. A reference housekeeping gene GADPH was amplified in the same plate and equivalent to all samples. The mRNA expression was measured in terms of the corresponding Threshold cycle (C_{T}) value and then normalized to GADPH expression in each experiment. The data was collected using Allelic Discrimination Assay on an ABI Prism 9700HT Sequence Detection System Instrument (Applied Biosystems) and exported to Microsoft Excel application for data analysis. The relative quantification was performed using the formula 2^{ΔΔCT}, where ΔΔC_{T}=C_{T (case group)}−C_{T (control group)}.

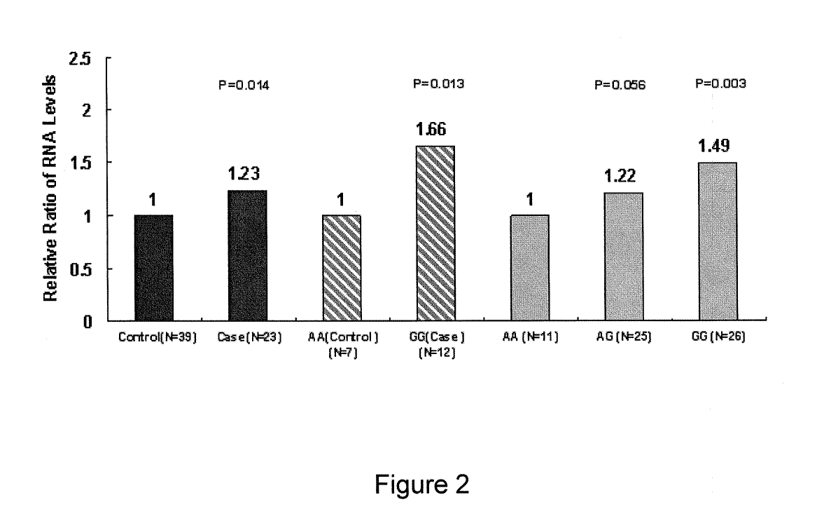

The ALPK1 expression differences between patient group and control group were analyzed using an independent sample t-test. The result with a p-value of less than 0.05 (p<0.05) was considered significant. Referring to FIG. 2, the relative ratio of mRNA levels was increased significantly in gout cases. Specifically, the ALPK1 mRNA level in gout cases was found to be 1.23 fold higher than those control cases (p=0.014). Furthermore, the relative ratio of mRNA level in gout patients having GG allele of rs231247 was 1.66 fold higher than controls having AA alleles (p=0.013). And the relative ratio of mRNA level in genotype GG and GA was respectively 1.49 and 1.22 fold higher than genotype AA (P=0.003 and 0.056).

### EXAMPLE 7

### Uric Acid Concentration and Fractional Excretion of Uric Acid (FEUA) Determinations

One hundred and ten unrelated individuals including 64 males (40 gout and 24 control subjects) and 46 females (10 gout and 36 control subjects) were recruited and phenotype. And 66% of subjects from the same community had chronic tophaceous gout. Creatinine and urate concentration were measured from the overnight urine sample in 24-hour for gouty subjects and 12-hour for controls using an automated analyzer (Beckman LX-20, Palo Alto, Calif., USA), and calculated for the appropriate fractional excretion of uric acid.

About 70% of daily urate disposal occurs via the kidneys, and in 5-25% of the human population, impaired renal excretion leads to hyperuricemia. As shown in Table 8, the role of rs231247 in the determination of uric acid concentrations and FEUA % was investigated to find that the rs231247 GG genotype was associated with high serum uric acid levels (8.25±0.10 mg/dl, p=0.017) and rs231247 GG genotype was associated with lower FEUA (5.02%, p=0.019). SNPs rs231247 was associated with a 19% decrease in proportion mean of FEUA (G/A allele, p=0.007). As the risk estimate for each one-locus allele combination was compared with the baseline of no risk allele (G at rs231247), a 2.09% to 7.24% increase of uric acid concentrations in circulating uric acid concentrations with ALPK1 gene and a 13.91% to 22.20% decrease in proportion mean of FEUA with ALPK1 gene were observed. Therefore, the above data also suggested that ALPK1 genetic variations may either through overproduction as well as underexcretion of urate to promote the occurrence of hyperuricemia/gout.

Various publications, patents, published patent applications are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed herein.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 1

| | | Five additional | | | |
|---|---|---|---|---|---|
| | | STRPs‡ | | SNPs of ALPK1 | |
| | | | Covariate•• | | Covariate |
| Markers | Location(cM | LOD | LOD | LOD | LOD |
| GATA2F11 | 104.90 | 0.83 | 1.77 | 0.75 | 1.52 |
| GATA62A12 | 114.00 | 2.39 | 3.43 | 1.92 | 2.83 |
| AFMa302wb5 | 116.00 | 3.68 | 4.91 | 2.33 | 3.36 |
| AFM164tf6* | 117.00 | 4.84* | 6.02 | 3.30 | 4.19 |
| rs231247 | (117.00) | — | — | 5.34† | 6.29 |
| APM186xb4 | 120.00 | 4.46 | 5.67 | 2.81 | 3.65 |
| AFM319yg9 | 121.00 | 1.28 | 2.34 | 1.71 | 3.02 |
| AFM151xc3 | 124.00 | 3.31 | 4.79 | 3.79 | 5.37 |
| TAGA006 | 124.60 | 3.49 | 5.00 | 3.97 | 5.57 |
| ATA26B08 | 130.00 | 1.81 | 3.09 | 2.15 | 3.56 |

*The most significant markers of five additional STRPs; Empirical p value = 1.54 •• 10⁻⁵
†The most significant SNP of ALPK1; Empirical p value = 6.40 •• 10⁻⁶
‡STRPs: Short tandem repeat polymorphisms; SNPs: Single-nucleotide polymorphisms; LOD: logarithm of odds.
••Covariate: Adjusting for alcohol consumption in the conditional-logistic model.

TABLE 2

| SEQ ID | | Risk | Gout | Control | | | | |
|---|---|---|---|---|---|---|---|---|
| NO: | SNP | allele | Frequency | Frequency | OR (95% CI) | p for genotype | p for allele | p for trend |
| 1 | rs916868 | C | 0.68 | 0.65 | 1.16 (0.95-1.42) | 3.00 •• 10⁻¹ | 1.44 •• 10⁻¹ | 1.45 •• 10⁻¹ |
| 2 | rs9994944 | G | 0.67 | 0.64 | 1.17 (1.00-1.37) | 1.63 •• 10⁻¹ | 5.53 •• 10⁻² | 5.82 •• 10⁻² |
| 3 | rs2074388 (D > G) | A | 0.59 | 0.53 | 1.28 (1.10-1.49) | 5.01 •• 10⁻³ | 1.35 •• 10⁻³ | 1.46 •• 10⁻³ |
| 4 | rs13148353 (R > H) | G | 0.61 | 0.56 | 1.22 (1.04-1.42) | 3.38 •• 10⁻² | 1.33 •• 10⁻² | 1.49 •• 10⁻² |
| 5 | rs2074379 (I > M) | A | 0.61 | 0.55 | 1.28 (1.06-1.55) | 3.21 •• 10⁻² | 1.03 •• 10⁻² | 1.24 •• 10⁻² |
| 6 | rs11726117 (T > M) | C | 0.62 | 0.55 | 1.30 (1.12-1.52) | 2.15 •• 10⁻³ | 7.50 •• 10⁻⁴ | 1.35 •• 10⁻³ |
| 7 | rs6841595 | C | 0.63 | 0.56 | 1.30 (1.07-1.57) | 3.23 •• 10⁻² | 7.64 •• 10⁻³ | 1.05 •• 10⁻² |
| 8 | rs11098156 | T | 0.20 | 0.16 | 1.33 (1.06-1.66) | 3.29 •• 10⁻² | 1.21 •• 10⁻² | 1.64 •• 10⁻² |
| 9 | rs231247 (R > R) | G | 0.62 | 0.54 | 1.36 (1.17-1.59) | 4.60 •• 10⁻⁴ | 8.00 •• 10⁻⁵ | 9.00 •• 10⁻⁵ |
| 10 | rs55840220 (T > T)‡ | A | 0.18 | 0.11 | 1.82 (1.47-2.26) | 3.32 •• 10⁻⁸ | 8.67 •• 10⁻⁹ | 9.51 •• 10⁻⁸ |
| 11 | rs231253 | G | 0.61 | 0.54 | 1.31 (1.12-1.53) | 3.30 •• 10⁻³ | 7.50 •• 10⁻⁴ | 8.90 •• 10⁻⁴ |
| 12 | rs960583 | A | 0.21 | 0.17 | 1.31 (1.08-1.59) | 1.91 •• 10⁻² | 6.01 •• 10⁻³ | 6.74 •• 10⁻³ |

*The risks associated with each SNP were estimated by allelic odds ratio (OR) using unconditional logistic regression, and the associated 95% confidence intervals (CI).
†P value: The p value after permutation analysis with 100,000 permutations by SAS/GENETIC software.
‡Hardy-Weinberg Equilibrium violation in Dayan replication.
••‘A’ denotes risk allele, ‘a’ non-risk allele

TABLE 3

| | Gout (%) | Control (%) | OR (95% CI)† | p‡ |
|---|---|---|---|---|
| ALPK1 rs231247 | | | | |
| Sequole tribe family-based | | | | |
| (92/61) | | | | |
| AA | 11 (12) | 14 (23) | 1.00 | |
| AG | 36 (39) | 30 (49) | 1.53 (0.61-3.86) | |
| GG | 45 (49) | 17 (28) | 3.37 (1.28-8.86) | 2.26 • 10⁻² |
| A | 58 (32) | 58 (48) | 1.00 | |
| G | 126 (68) | 64 (52) | 1.97 (1.23-3.16) | 5.59 • 10⁻³ |
| Dayan tribe for replication | | | | |
| (437/887) | | | | |
| AA | 72 (16) | 193 (22) | 1.00 | |
| AG | 202 (46) | 423 (48) | 1.28 (0.93-1.76) | |
| GG | 163 (37) | 271 (31) | 1.61 (1.16-2.25) | 1.56 • 10⁻² |
| A | 346 (40) | 809 (46) | 1.00 | |
| G | 528 (60) | 965 (54) | 1.28 (1.09-1.51) | 3.62 • 10⁻³ |
| Combined (529/948) | | | | |
| AA | 83 (16) | 207 (22) | 1.00 | |
| AG | 238 (45) | 453 (48) | 1.31 (0.97- | 5.00 • • 10⁻⁵ |
| GG | 208 (39) | 288 (30) | 1.80 (1.32- | 460 • • 10⁻⁴ |
| A | 404 (38) | 867 (46) | 1.00 | |
| G | 654 (62) | 1029 (54) | 1.36 (1.17- | 5.00 • • 10⁻⁵ |

†The risks associated with each SNP were estimated by genotypic and allelic odds ratio(OR) using logistic regression, and the associated 95% CI.
‡P value: The p value after permutation analysis with 100,000 permutations by SAS/GENETIC software.

TABLE 4

| SEQ ID | | | | Hyperuricemia | Controls | | Chi- | |
|---|---|---|---|---|---|---|---|---|
| NO: | SNP | Base pair | Minor allele | frequency | frequency | Major allele | square | p-value† |
| 1 | rs916868 | 113566163 | T | 0.33 | 0.37 | C | 2.18 | 0.364 |
| 2 | rs9994944 | 113566945 | A | 0.34 | 0.38 | G | 3.03 | 0.058 |
| 3 | rs2074388 (D > G) | 113571846 | G | 0.44 | 0.49 | A | 5.61 | 0.018 |
| 4 | rs13148353 (R > H) | 113572077 | A | 0.41 | 0.45 | G | 4.14 | 0.049 |
| 5 | rs2074379 (I > M) | 113572348 | G | 0.41 | 0.48 | A | 7.27 | 0.011 |
| 6 | rs11726117 (T > M) | 113572734 | T | 0.42 | 0.44 | C | 1.88 | 0.172 |
| 7 | rs6841595 | 113573291 | A | 0.39 | 0.46 | C | 5.85 | 0.021 |
| 8 | rs11098156 | 113575944 | T | 0.19 | 0.15 | G | 3.75 | 0.101 |
| 9 | rs231247 (R > R) | 113579152 | A | 0.42 | 0.45 | G | 2.42 | 0.121 |
| 10 | rs55840220 (T > T) | 113580374 | A | 0.14 | 0.11 | G | 3.30 | 0.113 |
| 11 | rs231253 | 113582071 | C | 0.42 | 0.47 | G | 4.37 | 0.042 |
| 12 | rs960583 | 113582497 | A | 0.19 | 0.17 | G | 1.37 | 0.286 |

*Hyperuricaemia was defined as serum uric acid >7 mg/dL for males and >6 mg/dL for females.
†P-value: The allelic p value after permutation analysis with 100,000 permutations by PLINK v1.00 software.

TABLE 5

| •• Genes/ | | | Drinking | |
|---|---|---|---|---|
| genotypes | Nondrinker | | •• Gout/ | |
| •• | Gout/Control | aOR (95% CI)* | Controls | aOR (95% CI) |
| ALPK1 rs231247 | | | | |
| AA | 17/90 | 1.00 | 50/102 | 2.45 (1.25-4.79) |
| AG | 46/213 | 1.22 (0.63-2.36) | 134/211 | 2.95 (1.60-5.43) |
| GG | 42/129 | 1.35 (0.69-2.66) | 127/126 | 4.30 (2.30-8.04) |

*aOR(Adjusted odds ratio) were adjusted for age, gender and familial aggregation in multiple logistic regression models, and the associated 95% confidence intervals (CI).

TABLE 6

| | |
|---|---|
| Nondrinker | Drinking |

| Genes/ | Gout/ | | •• Gout/ | |
|---|---|---|---|---|
| allele | Control | aOR (95% CI) | Control | aOR (95% CI) |
| ALPK1 | | | | |
| rs231247 | | | | |
| A | 80/393 | 1.00 | 234/415 | 2.42 (1.76-3.32) |
| G | 130/471 | 1.16 (0.83-1.62) | 388/463 | 3.31 (2.45-4.47) |

*OR (odds ratio) were adjusted for age, gender and familial aggregation in multiple logistic regression models, and the associated 95% confidence intervals (CI).

TABLE 7

| | | | | | |
|---|---|---|---|---|---|
| | | | | Chi- | |
| Haplotype | Gout % | Controls % | OR (95% CI) | Square | p-value |

| | | | | | |
|---|---|---|---|---|---|
| ALPK1(1066/1858) | | | | | |
| rs2074388/rs13148 | | | | | |
| 353/rs11726117/rs | | | | | |
| 231247 | | | | | |
| AGCG | 56 | 49 | 1.29 (1.10− | 15.30 | 8.00 · · 10⁻⁴ |
| GGCG | 2 | 2 | 1.29 (0.77− | 0.29 | 0.999 |
| GACA | 2 | 2 | 1.07 (0.61− | 0.01 | 1.000 |
| GGTA | 0.4 | 2 | 0.19 (0.07− | 14.00 | 1.50 · · 10⁻³ |
| AGTG | 1 | 1 | 0.91 (0.44− | 0.36 | 0.998 |
| GATG | 1 | 1 | 1.13 (0.55− | 0.00 | 1.000 |
| GATA | 35 | 39 | 1.00 | 4.92 | 0.196 |

*ALPK1, at risk haplotype (AGG) analysis indicates that 3 missense coding SNPs (rs2074388 A > G, rs13148353 G > A, and rs11726117 C > T) and one nonsense rs231247 G > A.
† P-value: The p value after permutation analysis with 100,000 permutations by Haploview 4.0 software.

TABLE 8

| | | | | Proportion | |
|---|---|---|---|---|---|
| | Mean trait value | | Difference | mean change | Bonferroni |
| | (SE) by allele | | mean | (%)† | p-value |
| Uric acid | | | | | |
| (n = 450/962) | | | | | |
| rs231247 | G | A | G/A | | |
| Gout | 10.07 (0.08) | 9.98 (0.10) | 0.08 (0.13) | 0.89 | 0.655 |
| Control | 7.10 (0.06) | 7.06 (0.06) | 0.04 (0.08) | 0.56 | 0.508 |
| All | 8.10 (0.05) | 7.92 (0.06) | 0.18 (0.08) | 2.22 | 0.028 |
| FEUA % | | | | | |
| (n = 50/60) | | | | | |
| rs231247 | G | A | G/A | | |
| Gout | 4.48 (0.21) | 4.71 (0.35) | −0.23 (0.36) | −5.13 | 0.526 |
| Control | 6.55 (0.30) | 7.71 (0.38) | −1.16 (0.52) | −17.71 | 0.028 |
| All | 5.57 (0.24) | 6.61 (0.30) | −1.04 (0.39) | −18.67 | 0.007 |

*Difference mean: Risk allele mean against reference mean (rs231247 G/A) was adjusted for age and sex after Bonferroni correction using mixed models.
†Proportion mean change (%): (Risk allele-reference allele) •• risk allele •• 100%

That which is claimed:

1. A method of identifying a human subject having an elevated risk of gout and/or hyperuricemia, the method comprising detecting the occurrence of at least one single nucleotide polymorphism (SNP) in an ALPK1 gene in a biological sample from the human subject, wherein the at least one SNP is selected from the group consisting of:
(a) a polymorphism comprising “C” at the nucleotide corresponding to nucleotide 15 of SEQ ID NO:1 (rs916868) or “G” at the nucleotide corresponding to nucleotide 15 of the corresponding minus strand of SEQ ID NO:1;
(b) a polymorphism comprising “G” at the nucleotide corresponding to nucleotide 15 of SEQ ID NO:2 (rs9994944) or “C” at the nucleotide corresponding to nucleotide 17 of the corresponding minus strand of SEQ ID NO:2;
(c) a polymorphism comprising “A” at the nucleotide corresponding to nucleotide 15 of SEQ ID NO:3 (rs2074388) or “T” at the nucleotide corresponding to nucleotide 16 of the corresponding minus strand of SEQ ID NO:3;
(d) a polymorphism comprising “G” at the nucleotide corresponding to nucleotide 16 of SEQ ID NO:4 (rs13148353) or “C” at the nucleotide corresponding to nucleotide 15 of the corresponding minus strand of SEQ ID NO:4;
(e) a polymorphism comprising “A” at the nucleotide corresponding to nucleotide 15 of SEQ ID NO:5 (rs2074379) or “T” at the nucleotide corresponding to nucleotide 16 of the corresponding minus strand of SEQ ID NO:5;
(f) a polymorphism comprising “C” at the nucleotide corresponding to nucleotide 15 of SEQ ID NO:6 (rs11726117) or “G” at the nucleotide corresponding to nucleotide 17 of the corresponding minus strand of SEQ ID NO:6;
(g) a polymorphism comprising “C” at the nucleotide corresponding to nucleotide 16 of SEQ ID NO:7 (rs6841595) or “G” at the nucleotide corresponding to nucleotide 15 of the corresponding minus strand of SEQ ID NO:7;
(h) a polymorphism comprising “T” at the nucleotide corresponding to nucleotide 15 of SEQ ID NO:8 (rs11098156) or “A” at the nucleotide corresponding to nucleotide 16 of the corresponding minus strand of SEQ ID NO:8;
(i) a polymorphism comprising “G” at the nucleotide corresponding to nucleotide 15 of SEQ ID NO:9 (rs231247) or “C” at the nucleotide corresponding to nucleotide 16 of the corresponding minus strand of SEQ ID NO:9;
(j) a polymorphism comprising “A” at the nucleotide corresponding to nucleotide 16 of SEQ ID NO:10 (rs55840220) or “T” at the nucleotide corresponding to nucleotide 15 of the corresponding minus strand of SEQ ID NO:10;
(k) a polymorphism comprising “G” at the nucleotide corresponding to nucleotide 16 of SEQ ID NO:11 (rs231253) or “C” at the nucleotide corresponding to nucleotide 15 of the corresponding minus strand of SEQ ID NO:11; and
(l) a polymorphism comprising “A” at the nucleotide corresponding to nucleotide 15 of SEQ ID NO:12 (rs960583) or “T” at the nucleotide corresponding to nucleotide 16 of the corresponding minus strand of SEQ ID NO:12;
wherein the occurrence of the at least one SNP is indicative of an elevated risk of gout and/or hyperuricemia in the human subject.

2. The method of claim 1, wherein the at least one SNP in the ALPK1 gene is the polymorphism comprising “G” at the nucleotide corresponding to nucleotide 15 of SEQ ID NO:9 (rs231247) or “C” at the nucleotide corresponding to nucleotide 16 of the corresponding minus strand of SEQ ID NO:9.

3. The method of claim 1 further comprising determining whether the human subject is heterozygous or homozygous for the at least one SNP.

4. The method of claim 1 further comprising determining alcohol consumption habit, high purine diet, age or gender of the human subject.

5. The method of claim 1 further comprising determining alcohol consumption habit of the human subject.

6. The method of claim 1 further comprising determining uric acid excretion of the human subject.

7. The method of claim 1, comprising detecting whether the human subject is homozygous for a SNP comprising “G” at the nucleotide corresponding to nucleotide 15 of SEQ ID NO:9 (rs231247) or “C” at the nucleotide corresponding to nucleotide 16 of the corresponding minus strand of SEQ ID NO:9, detecting whether the human subject has an alcohol consumption habit and detecting the uric acid excretion of the human subject.

8. A method of identifying a human subject having an elevated risk of gout and/or hyperuricemia, the method comprising determining the expression level of an ALPK1 gene in a biological sample from the human subject, wherein an elevated expression level of the ALPK1 gene is indicative of an elevated risk of gout and/or hyperuricemia in the human subject.

9. The method of claim 8, wherein the expression level of an ALPK1 gene is determined by reverse transcription polymerase chain reaction (RT-PCR).

10. The method of claim 8 further comprising determining alcohol consumption habit of the human subject.

11. The method of claim 8 further comprising determining uric acid excretion of the human subject.

12. A kit for performing a diagnostic test to identify a human subject having an elevated risk of gout and/or hyperuricemia, the kit comprising:
(a) at least one pair of primers designed for polymerase chain reaction amplification of a target polynucleotide sequence comprising a SNP in an ALPK1 gene in a biological sample from a human subject, wherein the SNP is selected from the group consisting of:
a polymorphism comprising “C” at the nucleotide corresponding to nucleotide 15 of SEQ ID NO:1 (rs916868) or “G” at the nucleotide corresponding to nucleotide 15 of the corresponding minus strand of SEQ ID NO:1;
a polymorphism comprising “G” at the nucleotide corresponding to nucleotide 15 of SEQ ID NO:2 (rs9994944) or “C” at the nucleotide corresponding to nucleotide 17 of the corresponding minus strand of SEQ ID NO:2;
a polymorphism comprising “A” at the nucleotide corresponding to nucleotide 15 of SEQ ID NO:3 (rs2074388) or “T” at the nucleotide corresponding to nucleotide 16 of the corresponding minus strand of SEQ ID NO:3;
a polymorphism comprising “G” at the nucleotide corresponding to nucleotide 16 of SEQ ID NO:4 (rs13148353) or “C” at the nucleotide corresponding to nucleotide 15 of the corresponding minus strand of SEQ ID NO:4;
a polymorphism comprising “A” at the nucleotide corresponding to nucleotide 15 of SEQ ID NO:5 (rs2074379) or “T” at the nucleotide corresponding to nucleotide 16 of the corresponding minus strand of SEQ ID NO:5;
a polymorphism comprising “C” at the nucleotide corresponding to nucleotide 15 of SEQ ID NO:6 (rs11726117) or “G” at the nucleotide corresponding to nucleotide 17 of the corresponding minus strand of SEQ ID NO:6;
a polymorphism comprising “C” at the nucleotide corresponding to nucleotide 16 of SEQ ID NO:7 (rs6841595) or “G” at the nucleotide corresponding to nucleotide 15 of the corresponding minus strand of SEQ ID NO:7;
a polymorphism comprising “T” at the nucleotide corresponding to nucleotide 15 of SEQ ID NO:8 (rs11098156) or “A” at the nucleotide corresponding to nucleotide 16 of the corresponding minus strand of SEQ ID NO:8;
a polymorphism comprising “G” at the nucleotide corresponding to nucleotide 15 of SEQ ID NO:9 (rs231247) or “C” at the nucleotide corresponding to nucleotide 16 of the corresponding minus strand of SEQ ID NO:9;
a polymorphism comprising “A” at the nucleotide corresponding to nucleotide 16 of SEQ ID NO:10 (rs55840220) or “T” at the nucleotide corresponding to nucleotide 15 of the corresponding minus strand of SEQ ID NO:10;
a polymorphism comprising “G” at the nucleotide corresponding to nucleotide 16 of SEQ ID NO:11 (rs231253) or “C” at the nucleotide corresponding to nucleotide 15 of the corresponding minus strand of SEQ ID NO:11; and
a polymorphism comprising “A” at the nucleotide corresponding to nucleotide 15 of SEQ ID NO:12 (rs960583) or “T” at the nucleotide corresponding to nucleotide 16 of the corresponding minus strand of SEQ ID NO:12; and
(b) instructions for performing the diagnostic test.

13. The kit of claim 11, further comprising at least one detection probe for the target polynucleotide sequence comprising the SNP.

14. A kit for performing a diagnostic test to identify a human subject having an elevated risk of gout and/or hyperuricemia, the kit comprising:
(a) at least one pair of primers designed for determination of the expression level of an ALPK1 gene in a biological sample from a human subject by real time reverse transcription polymerase chain reaction; and
(b) instructions for performing the diagnostic test.

15. The kit of claim 14, further comprising at least one detection probe for a complementary DNA of the ALPK1 gene.

16. A method of selecting a compound useful for treating gout and/or hyperuricemia, the method comprising
(a) contacting a test compound in a buffering solution with a first polypeptide comprising a catalytic domain of an ALPK1 protein and a second polypeptide comprising a phosphorylation site for the ALPK1 protein on an OAT 1 protein or an OAT 3 protein;
(b) detecting a change in phosphorylation level of the second polypeptide as a result of the phosphorylation of the second polypeptide by the catalytic domain; and
(c) selecting the test compound by its ability to decrease the phosphorylation level as compared to a control measurement wherein only the buffering solution, and not the test compound, is contacted with the first and second polypeptides.

17. The method of claim 16, wherein the first polypeptide is a product of expression of a recombinant host cell for the first polypeptide.

18. The method of claim 16, wherein the second polypeptide is a product of expression of a recombinant host cell for the second polypeptide.

19. The method of claim 16, wherein the second polypeptide is associated with an isolated membrane.

20. The method of claim 16, wherein the second polypeptide is present on the surface of a cell.

21. The method of claim 16, wherein the first polypeptide comprises an amino acid sequence of SEQ ID NO:13.

22. The method of claim 16, wherein the second polypeptide comprises an amino acid sequence of SEQ ID NO:14 or SEQ ID NO:15

23. The method of claim 16, further comprising
(a) administering the test compound to an animal; and
(b) determining the effect of the test compound on a symptom related to gout and/or hyperuricemia in the animal.

24. A method of identifying a compound useful for treating gout and/or hyperuricemia, comprising:
(a) contacting a test compound with a host cell that expresses a gene operably linked to a regulatory sequence for ALPK1 gene;
(b) detecting a change in the expression level of the gene from the host cell; and
(c) selecting the test compound by its ability to decrease the expression level of the gene, as compared to a control measurement wherein only the buffering solution, not the test compound, is contacted with the host cell.

25. The method of claim 24, further comprising
(a) administering the test compound to an animal; and
(b) determining the effect of the test compound on a symptom related to gout and/or hyperuricemia in the animal.

26. The method of claim 24, wherein the gene is a reporter gene operably linked to the regulatory sequence for ALPK1 gene.

27. The method of claim 26, wherein the reporter gene encodes a protein selected from the group consisting of green fluorescent protein, β-galactosidase, luciferase, chloramphenicol acetyltransferase, β-glucuronidase, neomycin phosphotransferase, and guanine xanthine phosphoribosyl-transferase.

28. An isolated nucleic acid molecule comprising a SNP of ALPK1 gene, wherein the SNP is selected from the group consisting of:
(a) a polymorphism comprising “C” at the nucleotide corresponding to nucleotide 15 of SEQ ID NO:1 (rs916868) or “G” at the nucleotide corresponding to nucleotide 15 of the corresponding minus strand of SEQ ID NO:1;
(b) a polymorphism comprising “G” at the nucleotide corresponding to nucleotide 15 of SEQ ID NO:2 (rs9994944) or “C” at the nucleotide corresponding to nucleotide 17 of the corresponding minus strand of SEQ ID NO:2;
(c) a polymorphism comprising “A” at the nucleotide corresponding to nucleotide 15 of SEQ ID NO:3 (rs2074388) or “T” at the nucleotide corresponding to nucleotide 16 of the corresponding minus strand of SEQ ID NO:3;
(d) a polymorphism comprising “G” at the nucleotide corresponding to nucleotide 16 of SEQ ID NO:4 (rs13148353) or “C” at the nucleotide corresponding to nucleotide 15 of the corresponding minus strand of SEQ ID NO:4;
(e) a polymorphism comprising “A” at the nucleotide corresponding to nucleotide 15 of SEQ ID NO:5 (rs2074379) or “T” at the nucleotide corresponding to nucleotide 16 of the corresponding minus strand of SEQ ID NO:5;
(f) a polymorphism comprising “C” at the nucleotide corresponding to nucleotide 15 of SEQ ID NO:6 (rs11726117) or “G” at the nucleotide corresponding to nucleotide 17 of the corresponding minus strand of SEQ ID NO:6;
(g) a polymorphism comprising “C” at the nucleotide corresponding to nucleotide 16 of SEQ ID NO:7 (rs6841595) or “G” at the nucleotide corresponding to nucleotide 15 of the corresponding minus strand of SEQ ID NO:7;
(h) a polymorphism comprising “T” at the nucleotide corresponding to nucleotide 15 of SEQ ID NO:8 (rs11098156) or “A” at the nucleotide corresponding to nucleotide 16 of the corresponding minus strand of SEQ ID NO:8;
(i) a polymorphism comprising “G” at the nucleotide corresponding to nucleotide 15 of SEQ ID NO:9 (rs231247) or “C” at the nucleotide corresponding to nucleotide 16 of the corresponding minus strand of SEQ ID NO:9;
(j) a polymorphism comprising “A” at the nucleotide corresponding to nucleotide 16 of SEQ ID NO:10 (rs55840220) or “T” at the nucleotide corresponding to nucleotide 15 of the corresponding minus strand of SEQ ID NO:10;
(k) a polymorphism comprising “G” at the nucleotide corresponding to nucleotide 16 of SEQ ID NO:11 (rs231253) or “C” at the nucleotide corresponding to nucleotide 15 of the corresponding minus strand of SEQ ID NO:11; and
(l) a polymorphism comprising “A” at the nucleotide corresponding to nucleotide 15 of SEQ ID NO:12 (rs960583) or “T” at the nucleotide corresponding to nucleotide 16 of the corresponding minus strand of SEQ ID NO:12.

29. A collection of nucleic acid molecules, comprising two or more nucleic acid molecules of claim 28.

30. The collection of claim 29, wherein each of the two or more nucleic acid molecules comprises at least about 10 nucleotides.

31. The collection of claim 29, wherein the nucleic acid molecules of the collection are affixed to a solid surface in an array.

32. A method of identifying a human subject having an elevated risk of gout and/or hyperuricemia, the method comprising detecting the occurrence of at least two SNPs in an ALPK1 gene in a biological sample from the human subject, wherein the at least two SNPs are detected using a collection of claim 28.

33. A vector comprising the isolated nucleic acid molecule of claim 28.

34. A host cell comprising the vector of claim 33.
